(19) United States
(12) Patent Application Publication
(10) Pub. No.: US 2026/0358352 A1
(43) Pub. Date: Nov. 12, 2026

(54) DIAGNOSTIC AGENT AND METHOD FOR ALZHEIMER'S DISEASE

(71) Applicant: Temasek Life Sciences Laboratory Limited, Singapore (SG)

(72) Inventors: Chin Tong ONG, Singapore (SG); Zhen Kai NGIAN, Singapore (SG); Yow Yong TAN, Singapore (SG)

(21) Appl. No.: 19/108,102

(22) PCT Filed: Aug. 31, 2023
(86) PCT No.: PCT/SG2023/050599

§ 371 Date: Feb. 28, 2025

(30) Foreign Application Priority Data
Sep. 2, 2022 (SG) 10202250889Y

(51) Int. Cl. G01N 33/68; C07K 14/47; C07K 16/18; C12Q 1/6883

(52) U.S. Cl. CPC G01N 33/6896; C07K 14/47; C07K 16/18; C12Q 1/6883; C12Q 2600/158; G01N 2800/2821

(57) ABSTRACT

The invention relates to identification of an intron-retaining Tau splicing isoform as a novel Alzheimer's disease biomarker. Provided herein are polypeptides to generate binding molecules, such as antibodies specific for the Tau11i isoform, oligonucleotides and antibodies for use in methods for detecting the Tau11i isoform in a sample and methods for use in diagnosis for Alzheimer's disease.

### CROSS-REFERENCE TO RELATED APPLICATIONS

The present application is a filing under 35 U.S.C. 371 as the National Stage of International Application No. PCT/SG2023/050599, filed Aug. 31, 2023, entitled “DIAGNOSTIC AGENT AND METHOD FOR ALZHEIMER'S DISEASE,” which claims priority to Singapore Application No. 10202250889Y filed with the Intellectual Property Office of Singapore on Sep. 2, 2022, both of which are incorporated herein by reference in their entirety for all purposes.

### INCORPORATION BY REFERENCE OF MATERIAL IN XML FILE

This application incorporates by reference the Sequence Listing contained in the following XML file being submitted concurrently herewith:

File name: 4373-20600 SP105901USZ Sequence Listing ST26, created on Apr. 2, 2025; and having a file size of 40 KB. The information in the Sequence Listing is incorporated herein in its entirety for all purposes.

### FIELD OF THE INVENTION

The present invention relates to an intron-retaining Tau splicing isoform and methods of detecting the same. The invention also relates to agents, including oligonucleotides and antibodies, and methods of diagnosis for Alzheimer's disease.

### BACKGROUND OF THE INVENTION

Alzheimer's disease (AD) is a progressive dementia that starts with decrease of short-term memory and mild learning disability, develops higher brain dysfunction, particularly visuospatial agnosia, ideational apraxia, constructive apraxia and the like, and finally reaches movement disorder and so-called personality destruction. It is a familial and sporadic neurodegenerative disease that encompasses many risk factors such as genetic makeup and ageing. Synaptic and neuronal loss in AD is caused by interplay between the pathology of amyloid-β plaques and Tau neurofibrillary tangles with impairment of innate clearance pathways. The microtubule-associated protein Tau and its hyper-phosphorylated version form the main constituent of intracellular neurofibrillary tangles which is a hallmark of several dementias, including AD and frontotemporal dementia. This evidence forms the basis of a hypothesis for AD, wherein the intracellular accumulation of Tau leads to microtubule disassembly, dendritic spinal collapse, and degeneration of axons, malfunction in communication between neurons and cell death. Accumulated evidence indicates that epigenetic alterations and aberrant RNA splicing events at several AD susceptibility loci during ageing can predispose healthy brains to AD pathogenesis, with reports showing differential intron retention (IR) events at the Tau gene (Adusumalli et al., Aging Cell 18, e12928 (2019); Garcia-Escudero et al., Acta Neuropathol 142, 159-177 (2021); Trabzuni et al., Hum. Mol. Genet. 21, 4094-4103 (2012)); IR has been implicated in the pathogenesis of complex diseases such as cancers; but its association with AD remains unexplored. Accordingly, there is a need to elucidate the relationship between AD and intron-retaining Tau proteins, identify novel epitopes of pathological species of Tau and provide agents and methods useful for diagnosis of AD.

### SUMMARY OF THE INVENTION

The present invention aims to provide agents and methods for diagnosing Alzheimer's disease or identifying a subject at risk of developing Alzheimer's disease.

In a first aspect, there is provided an isolated or recombinant Tau11i-derived polypeptide comprising the following amino acid sequence:
(1) the amino acid sequence HKPGSPVEGEGWDGRVQGV, as set forth in SEQ ID NO: 14 or antigenic fragment thereof, or
(2) the amino acid sequence SPVEGEGWDGRVQG, as set forth in SEQ ID NO: 15, or
(3) an amino acid sequence resulting from substitution, deletion, addition or insertion of one or two or more amino acids in the amino acid sequence set forth in SEQ ID NO: 14 or SEQ ID NO: 15.

In some embodiments, the isolated or recombinant Tau11i-derived polypeptide may consist of amino acid sequence set forth in (1) or (2) with a cysteine residue conjugated to an end. Terminal cysteine is added to the peptide sequence to allow its conjugation to carrier proteins, which helps antibody production. In some embodiments, the isolated or recombinant Tau11i-derived polypeptide may consist of the amino acid sequence HKPGSPVEGEGWDGRVQGV-C set forth in SEQ ID NO: 1 or the amino acid sequence C-SPVEGEGWDGRVQG set forth in SEQ ID NO: 2.

In a second aspect, there is provided an isolated antibody or antigen-binding fragment thereof that specifically binds to the isolated or recombinant Tau11i-derived polypeptide comprising the amino acid sequence set forth in SEQ ID NO: 14 or antigenic fragment thereof, or SEQ ID NO: 15, or an amino acid sequence resulting from substitution, deletion, addition or insertion of one or two or more amino acids in the amino acid sequence set forth in SEQ ID NO: 14 or SEQ ID NO: 15. In some embodiments, the polypeptide in which the antibody or the antigen binding fragment thereof binds to consists of the amino acid sequence set forth in SEQ ID NO:1 or SEQ ID NO: 2.

In some embodiments, the isolated antibody or antigen-binding fragment thereof comprises a heavy chain variable region comprising the amino acid sequence shown as residues 1 to 136 of SEQ ID NO: 29 and/or a light chain variable region comprising the amino acid sequence shown as residues 1 to 132 of SEQ ID NO: 34. In some embodiments, the isolated antibody or antigen-binding fragment thereof comprises a heavy chain variable region comprising complementarity determining regions HCDR1 having an amino acid sequence set forth in SEQ ID NO: 31, HCDR2 having an amino acid sequence set forth in SEQ ID NO: 32, and HCDR3 having an amino acid sequence set forth in SEQ ID NO: 33, and/or a light chain variable region comprising complementarity determining regions LCDR1 having an amino acid sequence set forth in SEQ ID NO: 36, LCDR2 having an amino acid sequence set forth in SEQ ID NO:37, and LCDR3 having an amino acid sequence set forth in SEQ ID NO: 38.

In a third aspect, there is provided oligonucleotides for amplifying a polynucleotide sequence spanning the junction of exon 11 and intron 11 of a Tau gene. In some embodiments, the oligonucleotides consist of the nucleotide sequences set forth in SEQ ID NO: 7 and SEQ ID NO: 8.

In a fourth aspect, there is provided a kit comprising the antibody or antigen binding fragment thereof provided in the second aspect. In some embodiments, the kit further comprises the polypeptide of the first aspect.

In a fifth aspect, there is provided a polynucleic acid molecule encoding the polypeptide according to the first aspect, or the antibody or antigen binding fragment thereof according to the second aspect. In some embodiments, the polynucleic acid molecule is cDNA.

In a sixth aspect, there is provided a method of detecting the polypeptide according to the first aspect, wherein the method comprises contacting a sample suspected of comprising the polypeptide with the antibody or the antigen-binding fragment according to the second aspect. Preferably, the method is in vitro.

In a seventh aspect, there is provided a method for diagnosing Alzheimer's disease or identifying a subject at risk of developing Alzheimer's disease, wherein the method comprises:
(a) contacting a sample derived from the subject with the antibody or antigen binding fragment thereof according to the second aspect; or
(b) conducting a nucleic acid detection assay to assess the abundance of intron 11 retaining mRNA in a sample derived from the subject; or
(c) contacting a sample derived from the subject with a Tau11i epitope-specific aptamer that specifically binds to a polypeptide comprising the amino acid sequence set forth in any one of SEQ ID NOs: 1, 2, 13, 14 or 15.

In some embodiments, the sample is brain tissue. In some embodiments, the sample is cerebrospinal fluid (CSF). In some embodiments, the sample is blood plasma. In other embodiments, the sample is saliva, lacrimal fluid or urine. In some embodiments, the nucleic acid detection assay comprises amplifying a polynucleotide sequence spanning the junction of exon 11 and intron 11 of a Tau gene. In further embodiments, the amplification comprises using oligonucleotides having the nucleotide sequences as set forth in SEQ ID NOs: 7 and 8. In further embodiments, the nucleic acid detection assay is a PCR-based assay.

In an eighth aspect, there is provided a method of screening a candidate therapeutic or prophylactic drug for Alzheimer's disease, wherein the method comprises using any of the following as an index: (a) reduction of the amount of polypeptide-containing Tau (e.g. Tau11i as hereinafter described) in the body of an Alzheimer's disease subject or a subject at risk of developing Alzheimer's disease, or (b) inhibition of production of polypeptide-containing Tau (e.g. Tau11i as hereinafter described) in the body of an Alzheimer's disease subject or a subject at risk of developing Alzheimer's disease; wherein the Tau protein comprises the amino acid sequence of the polypeptide according to the first aspect.

In a ninth aspect, there is provided a method of preparing a Tau11i-specific antibody, wherein the method comprises: (a) injecting the polypeptide according to the first aspect into an animal to induce an immune response to the polypeptide; (b) isolating antibodies that bind to the polypeptide; (c) selecting antibodies of (b) that binds to Tau11i but do not bind to full length Tau thereby producing an Tau11i-specific antibody.

In a tenth aspect, there is provided a method of screening and/or selecting a Tau11i epitope-specific aptamer, the method comprises: (a) conducting systematic evolution of ligands by exponential enrichment (SELEX) technique to identify Tau11i epitope-specific aptamers, wherein the SELEX technique comprises providing a polypeptide comprising the amino acid sequence set forth in any one of SEQ ID NOs. 1, 2, 13, 14 or 15 as a target ligand.

In an eleventh aspect, there is provided an aptamer capable of binding to a polypeptide comprising the amino acid sequence set forth in any one of SEQ ID NOs. 1, 2, 13, 14 or 15.

### BRIEF DESCRIPTION OF THE FIGURES

The accompanying drawings illustrate disclosed embodiments and serve to explain the principles of the disclosed embodiments. It is to be understood, however, that the drawings are designed for purposes of illustration only, and not as a definition of the limits of the invention.

FIGS. 1A-1D show an increased retention of intron 11 of Tau gene in AD. (FIG. 1A) Intron 11 contains a premature stop codon (line 2: gagtcctggtgaggc . . . ) with a canonical polyadenylation site that is located 1 kb downstream. Arrows indicate qPCR primers. (FIG. 1B) Integrative Genomic Viewer of intron 11 from control (top two rows) and AD (bottom two rows) patients. (FIG. 1C) Left: Statistically significant higher intron retention (IR) is observed only in female AD dorsal lateral prefrontal cortex (n=34; right column) as compared to control (CT, n=32; left column). Each dot represents individual normalized IR ratio. Right: No differential expression in the Tau gene between CT (left column) and AD (right column) cohorts. (FIG. 1D) qPCR validation of intron 11 (relative to Tau exon1/exon2) and Tau expression (exon1/exon 2 and exon5/exon6 relative to GAPDH) from 3 distinct pairs of brain tissues from control (left column) and AD (right column) cohorts. “FL” and “TL” stand for frontal lobe and temporal lobe, respectively. Data presented as mean of triplicates±SD, p-value calculated by 2-tailed t-test.

FIGS. 2A-2D show that truncated Tau11i oligomerizes and is found in a Sarkosyl-insoluble fraction. (FIG. 2A) Schematic of Flag-tag full-length and truncated Tau proteins with specific antibody recognition sites. Amino acids encoded by intron 11 are represented in light grey text. pT181: phosphor-Tau (T181), Tau4R: 275-291, Tau46: 404-421, AT8: phosphor-Tau (S202/T205). (FIGS. 2B-2C) Immunoblot and quantification of Tau11i HMW species in (B) 293 cells transfected with Tau441 (left well) or Tau11i (right well); and (C) Neuronal stem cells (NSC) (left lane) that expressed either Tau441 (middle lane) or Tau11i (right lane) proteins. Data presented as mean±SEM. (FIG. 2D) Immunoblot showed elevated Sarkosyl-insoluble fraction of Tau11i in 293T cells (n=2; top blot, right lane), and Day 44 Neurons (bottom blot) (n=3 each) expressing Flag-Tau441 or Flag-Tau11i. Quantification of the relative level of Sarkosyl-insoluble Tau to total lysate is presented as mean±SD. All p-value was calculated by 2-tailed t-test.

FIGS. 3A-3C show an elevated level of Tau11i in different AD brain tissues. (FIG. 3A) Immunoblot of control (“C” lanes) and AD (“A” lanes) temporal lobe, amygdala (AMYDG.), parietal lobe (PARIETAL), frontal lobe and hippocampus showed enrichment of Tau11i (~55 kDa white arrowhead) in AD samples. Samples were also immunoblotted with AT8 and actin antibodies. Each number depicts a unique human subject. Quantification of 7 pairs of control and AD samples with data presented as mean±SD. The level of Tau11i in control for each brain region was set as 1. Sample quantified was underlined. The p-value was calculated by paired 2-tailed t-test. (FIG. 3B) Immunoblots showed that Tau11i is found in Sarkosyl-insoluble fraction (white arrow) in AD hippocampus and temporal lobe. 10% of Total Lysate (Lys), 15% of soluble fraction (Sol.) and 50% of Sarkosyl-insoluble fraction (Insol.) were loaded. “Tech Rep” stands for technical replicate. (FIG. 3C) Immunofluorescence showed weak colocalization of Tau11i aggregates with Tau4R fibrils in AD temporal lobe. Scale bar represents 20 μm. Each circle represents one field. Data presented as mean±SD with p-value calculated by 2-tailed t-test.

FIGS. 4A-4C show that Tau11i protein associates poorly with the microtubule network and is more stable. (FIG. 4A) Images of Day 21 and Day 44 neurons expressing Tau441 or Tau11i stained with α-tubulin and Flag antibodies. Scale bar represents 50 μm. Quantification showed lower colocalization of Tau11i with α-tubulin as compared to Tau441. Each circle represents one field with filled circle from second biological replicate. Data presented as mean±SD. (FIG. 4B) Immunoblot using Flag and actin antibodies in 293T cells harvested on different day “D” after transfection. Quantification of Tau level (D7 relative to D3; Tau11i right bar) is presented as mean±SEM (n=5). (FIG. 4C) Immunoblot of Tau level after cycloheximide (CHX) treatment of 293T cells on Day 5 after transfection. “Rep” stands for replicate. Quantification of Tau level (8 h relative to 0 h) is presented as mean±SD where each circle represents one replicate (n=12; Tau11i on right). The p-value was calculated by 2-tailed t-test.

FIGS. 5A-5B depict retention of intron 11 of the Tau gene in other neurodegeneration and characterization of Tau11i protein. (FIG. 5A) Dot plot of normalized intron retention (IR) ratio from PD and PSP cohorts. Each dot represents individual value. CT column is healthy control and PD or PSP column labels diseased patient. Data is presented as mean±SD, p-value is calculated by 2-tailed t-test. Related to FIG. 1. (FIG. 5B) Immunoblot of 293T cells (top) and NSC (bottom) expressing either Flag-Tau441 (left well) or Flag-Tau11i (right well). HMW species is marked by white arrowhead. “Rep” stands for replicate. Related to FIG. 2.

FIGS. 6A-6B depict the results of immunofluorescence which show a higher level of Tau11i in AD temporal lobe (middle panels). (FIG. 6A) Image and quantification of Tau11i over DAPI positive cells is presented as mean±SD where each circle represents one field. P-value was calculated by 2-tailed t-test. (FIG. 6B) Immunoblot of control “C8” and AD “A1” temporal lobe. Related to FIG. 3C.

FIGS. 7A-7B show that Tau11i has a lower level of co-localization with α-tubulin as compared to Tau441 in mature neurons. Representative image from different biological replicate of (FIG. 7A) Day 21 and (FIG. 7B) Day 44 neurons. Scale bar is 100 μm. Quantification of Flag-Tau over α-tubulin is presented as mean±SD where each circle represents one field. P-value was calculated by 2-tailed t-test. “Rep” stands for replicate. Related to FIG. 4A.

FIGS. 8A-8B show that Tau11i has a weaker co-localization signal with α-tubulin in mature neurons and higher protein stability when compared to Tau441. (FIG. 8A) Representative images from two biological replicates of Day 44 neurons expressing either Flag-Tau441 or Flag-Tau11i. Scale bar is 100 μm. Quantification of Flag-Tau over α-tubulin is presented as mean±SD where each circle represents one field. P-value was calculated by 2-tailed t-test. Related to FIG. 4A. “Rep” stands for replicate. (FIG. 8B) Representative immuno-blots of 293T cells harvested on different day “D” after transfection with Flag and actin antibodies. Related to FIG. 4B.

FIGS. 9A-9B show the detection of Tau intron 11 retention in total RNA extracted from human plasma. (FIG. 9A) Schematic of experiment. Total RNA was extracted from 0.5 ml of control human plasma (i-DNA Biotech) using RNAzol (Sigma). After DNase I treatment to remove genomic DNA, reverse transcription was performed with random hexamer. cDNA was subjected to PCR and resolved on agarose gel. (FIG. 9B) Different primers were used to amplify the Tau gene and detect intron retention events previously identified in AD brains. The HBB gene is highly expressed, therefore we used it as positive control and tested for the presence of genomic contamination. HADC2 is not present in blood plasma and was used as a negative control. Amplification of Tau intron 11 from plasma RNA is shown in lane 3.

FIGS. 10A-10B show that Tau11i is detectable in human plasma with an anti-Tau11i antibody. (FIG. 10A) 293 cells or 293 cells transfected with Flag-Tau11i were lysed and Co-IP with rabbit anti-Tau11i antibody, which was generated with antigen peptide consisting of the amino acid sequence set forth in SEQ ID NO: 15. The Co-IP beads were boiled and immunoblotted with Flag antibody. Arrow heads show immunoprecipitated Tau11i protein. (FIG. 10B) Pre-cleared control human plasma (i-DNA Biotech) was Co-IP with Tau11i antibody, resolved in SDS-PAGE and analyzed by LC-MS/MS after in-gel digestion and column clean-up.

FIGS. 11A-11B depict the detection of Tau11i by ELIZA. (FIG. 11A) Schematic of Flag-Tau441 & Flag-Tau11i peptide (which were purified from 293T cells). (FIG. 11B) Optimization of ELISA. In experiment 1, wells were coated with Tau11i or Tau441 peptide & probed with Tau11i antibody. In experiment 2, Tau peptide-coated wells were probed with either Tau11i, IgG or phospho-Tau181 antibodies. In experiment 3, Tau11i antibody was coated onto plates with the addition of either Flag-Tau11i or Flag-Tau441 peptides. An anti-Flag antibody was used for detection. Results show the anti-Tau11i antibody having much higher specificity for Tau11i compared with Tau441, which was around negative control level.

FIGS. 12A-12B depict the detection of Tau11i by monoclonal antibody 8A12. (FIG. 12A) Schematic of Flag-Tau441 & Flag-Tau11i peptide. (FIG. 12B) Immunoblot of total lysates from 293 cells and neuronal stem cells (NSC) that expressed either Flag-Tau441 or Flag-Tau11i protein with Flag or monoclonal Tau11i antibody (8A12). PICO indicates lower exposure whereas DURA indicates higher exposure.

### DETAILED DESCRIPTION OF THE INVENTION

The following detailed description refers to, by way of illustration, specific details and embodiments in which the invention may be practiced. These embodiments are described in sufficient detail to enable those skilled in the art to practice the invention. Other embodiments may be utilized and structural, and logical changes may be made without departing from the scope of the invention. The various embodiments are not necessarily mutually exclusive, as some embodiments can be combined with one or more other embodiments to form new embodiments.

Bibliographic references mentioned in the present specification are for convenience listed in the form of a list of references and added at the end of the examples. The whole content of such bibliographic references is herein incorporated by reference but their mention in the specification does not imply that they form part of the common general knowledge.

### DEFINITIONS

For convenience, certain terms employed in the specification, examples and appended claims are collected here.

In general, technical, scientific and medical terminologies used herein has the same meaning as understood by those skilled in the art to which this invention belongs. Further, the following technical comments and definitions are provided. These definitions should in no way limit the scope of the present invention to those terms alone, but are put forth for a better understanding of the following description.

As used herein, the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, “Alzheimer's disease” or “AD” includes both familial Alzheimer's disease and sporadic Alzheimer's disease.

As used herein, the term “comprising” or “including” is to be interpreted as specifying the presence of the stated features, integers, steps or components as referred to, but does not preclude the presence or addition of one or more features, integers, steps or components, or groups thereof. However, in context with the present disclosure, the term “comprising” or “including” also includes “consisting of”. The variations of the word “comprising”, such as “comprise” and “comprises”, and “including”, such as “include” and “includes”, have correspondingly varied meanings.

As used herein, the term “subject” or “individual” or “patient” may be used interchangeably and refers to any subject, particularly a mammalian subject, e.g., a human patient, for whom diagnosis, prognosis, prevention, or therapy is desired.

As used herein, the term “sample” refers to any biological material obtained from a subject or patient. For example, a sample can comprise blood, cerebrospinal fluid (“CSF”), and/or urine. In other examples, a sample can comprise whole blood or plasma. A sample can also include a biopsy or tissue sample including neural tissue. In still other examples, a sample can comprise whole cells and/or a lysate of the cells. Blood samples can be collected by methods known in the art.

As used herein, “Tau” or “tau” refers to the native monomer form of Tau and is also used to generally identify other conformers of Tau, for example, oligomers or aggregates of Tau. It is also used to refer collectively to all types and forms of Tau.

As used herein, “monomeric Tau” or “Tau monomer” refers to completely solubilized Tau proteins without aggregated complexes in aqueous medium. “Aggregated Tau”, “oligomeric Tau” and “Tau oligomer” refers to multiple aggregated monomers of Tau peptides or proteins, or of Tau-like peptides/proteins, or of modified or truncated Tau peptides/proteins or of other derivates of Tau peptides/proteins forming oligomeric or polymeric structures which are insoluble or soluble both in vitro in aqueous medium and in vivo in the mammalian or human body more particularly in the brain, but particularly to multiple aggregated monomers of Tau or of modified or truncated Tau peptides/proteins or of derivatives thereof, which are insoluble or soluble in the mammalian or human body more particularly in the brain, respectively.

### Polynucleotide, Gene and Nucleic Acid Detection

As used herein, the term “polynucleotide” or “polynucleic acid” may be used interchangeably and refers to a polymer comprising multiple nucleotide monomers (e.g., ribonucleotide monomers or deoxyribonucleotide monomers). The term includes, for example, genomic DNA, cDNA, RNA and DNA-RNA hybrid molecules. Polynucleic acid molecules/polynucleotides can be naturally occurring, recombinant, or synthetic. The term “polynucleotide” is intended to encompass a singular nucleic acid as well as plural nucleic acids, and refers to an isolated nucleic acid molecule or construct, e.g., messenger RNA (mRNA) or plasmid DNA (pDNA), or PCR amplicons. A polynucleotide can comprise a conventional phosphodiester bond or a non-conventional bond (e.g., an amide bond, such as found in peptide nucleic acids (PNA)). By “isolated” polynucleotide is intended a nucleic acid molecule, DNA or RNA, which has been removed from its native environment.

As used herein, the term “gene” refers to a nucleic acid (e.g., DNA or RNA) sequence that comprises coding sequences necessary for the production of an RNA, or of a polypeptide or its precursor. A functional polypeptide may be encoded by a full-length coding sequence or by any portion of the coding sequence as long as the desired activity or functional properties (e.g., enzymatic activity, ligand binding, signal transduction, etc.) of the polypeptide are retained. The term “portion” when used in reference to a gene may refer to fragments of that gene. The fragments may range in size from a few nucleotides to the entire gene sequence minus one nucleotide.

The term “gene” also encompasses the coding regions of a structural gene and includes sequences located adjacent to the coding region on both the 5′ and 3′ ends, e.g., for a distance of about 1 kb on either end, such that the gene corresponds to the length of the full-length mRNA (e.g., comprising coding, regulatory, structural and other sequences). The sequences that are located 5′ of the coding region and that are present on the mRNA are referred to as 5′ non-translated or untranslated sequences. The sequences that are located 3′ or downstream of the coding region and that are present on the mRNA are referred to as 3′ non-translated or 3′ untranslated sequences. The term “gene” encompasses both cDNA and genomic forms of a gene. In some organisms (e.g., eukaryotes), a genomic form or clone of a gene contains the coding region interrupted with non-coding sequences termed “introns” or “intervening regions” or “intervening sequences.” Introns are segments of a gene that are transcribed into nuclear RNA; introns may contain regulatory elements such as enhancers. Introns are removed or “spliced out” from the nuclear or primary transcript; introns therefore are absent in the mRNA transcript. The mRNA functions during translation to specify the sequence or order of amino acids in a nascent polypeptide.

As used herein, the term “nucleic acid detection assay” refers to any method of determining the nucleotide composition of a nucleic acid of interest. Nucleic acid detection assay may include, but are not limited to, DNA/RNA sequencing methods, probe hybridization methods, structure specific cleavage assays (e.g., the INVADER assay, Hologic, Inc.; and are described, e.g., in U.S. Pat. Nos. 5,846,717, 5,985,557, 5,994,069, 6,001,567, 6,090,543, and 6,872,816; Lyamichev et al., Nat. Biotech., 17:292 (1999), Hall et al., Proc. Natl. Acad. Sci. USA, 97:8272 (2000), and US 2009/0253142); enzyme mismatch cleavage methods (e.g., Variagenics, U.S. Pat. Nos. 6,110,684, 5,958,692, 5,851,770); polymerase chain reaction (PCR); branched hybridization methods (e.g., Chiron, U.S. Pat. Nos. 5,849,481, 5,710,264, 5,124,246, and 5,624,802); rolling circle replication (e.g., U.S. Pat. Nos. 6,210,884, 6,183,960 and 6,235,502); NASBA (e.g., U.S. Pat. No. 5,409,818); molecular beacon technology (e.g., U.S. Pat. No. 6,150,097); E-sensor technology (Motorola, U.S. Pat. Nos. 6,248,229, 6,221,583, 6,013,170, and 6,063,573); cycling probe technology (e.g., U.S. Pat. Nos. 5,403,711, 5,011,769, and 5,660,988); Dade Behring signal amplification methods (e.g., U.S. Pat. Nos. 6,121,001, 6,110,677, 5,914,230, 5,882,867, and 5,792,614); ligase chain reaction (e.g., Barany, F., Proc. Natl. Acad. Sci. USA 88, 189-93 (1991)); sandwich hybridization methods (e.g., U.S. Pat. No. 5,288,609); and Specific High-sensitivity Enzymatic Reporter un-LOCKing (e.g., Gootenberg J. S. et al., Science 356, 438-442 (2017)).

As used herein, the term “amplification of nucleic acids” generally refers to the production of multiple copies of a polynucleotide, or a portion of the polynucleotide, typically starting from a small amount of the polynucleotide (e.g., a single polynucleotide molecule, 10 to 100 copies of a polynucleotide molecule, which may or may not be exactly the same), where the amplification products or amplicons are generally detectable. Amplification of polynucleotides encompasses a variety of chemical and enzymatic processes. The generation of multiple DNA copies from one or a few copies of a target or template DNA molecule during a polymerase chain reaction (PCR) or a ligase chain reaction (LCR; see, e.g., U.S. Pat. No. 5,494,810) are forms of amplification. Additional types of amplification may include, but are not limited to, allele-specific PCR (see, e.g., U.S. Pat. No. 5,639,611), assembly PCR (see, e.g., U.S. Pat. No. 5,965,408), helicase-dependent amplification (see, e.g., U.S. Pat. No. 7,662,594), Hot-start PCR (see, e.g., U.S. Pat. Nos. 5,773,258 and 5,338,671), intersequence-specific PCR, inverse PCR (see, e.g., Triglia, et al., Nucleic Acids Res., 16:8186 (1988)), ligation-mediated PCR (see, e.g., Guilfoyle, R. et al., Nucleic Acids Res., 25:1854-1858 (1997); U.S. Pat. No. 5,508,169), methylation-specific PCR (see, e.g., Herman, et al., Proc. Natl. Acad. Sci. USA 93 (18) 9821-9826 (1996)), miniprimer PCR, multiplex ligation-dependent probe amplification (see, e.g., Schouten, et al. Nucleic Acids Res. 30 (12): e57 (2002)), multiplex PCR (see, e.g., Chamberlain et al., Nucleic Acids Res. 16 (23) 11141-11156 (1998); Ballabio, et al., Hum. Genet. 84 (6) 571-573 (1990); Hayden, et al., BMC Genom. 9:80 (2008), nested PCR, overlap-extension PCR (see, e.g., Higuchi, et al., Nucleic Acids Res. 16 (15) 7351-7367 (1998)), real time PCR (see, e.g., Higuchi, et al., Biotechnol. 10:413-417 (1992); Higuchi, et al., Biotechnol. 11:1026-1030 (1993)), reverse transcription PCR (see, e.g., Bustin, S. A., Hum. Genet. 84, 571-573 (2000)), solid phase PCR, thermal asymmetric interlaced PCR, and Touchdown PCR (see, e.g., Don et al., Nucleic Acids Res. 19 (14) 4008 (1991); Roux, K. H., BioTechniques 16 (5) 812-814 (1994); Hecker, K. H., & Roux, K. H., BioTechniques 20 (3) 478-485 (1996)). Polynucleotide amplification also can be accomplished using digital PCR (see, e.g., Kalinina, et al., Nucleic Acids Res. 25; 1999-2004, (1997); Vogelstein B. and Kinzler K. W., Proc. Natl. Acad. Sci. USA. 96; 9236-41, (1999); International Patent Publication No. WO05023091A2; US Patent Application Publication No. 20070202525).

As used herein, the term “oligo” or “oligonucleotide” refers to short length single or double stranded polydeoxynucleotides which are chemically synthesized and then purified by known methods. Oligos may be used in nucleic acid amplification techniques where such oligos may alternatively be termed as primers.

### Polypeptides of the Invention

As used herein, the term “polypeptide” is intended to encompass a singular “polypeptide” as well as plural “polypeptides,” and refers to a polymer molecule of at least two amino acids (monomers) covalently linked by amide bonds (also known as peptide bonds). The term “polypeptide” refers to any chain or chains of two or more amino acids and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” can be used instead of, or interchangeably with any of these terms. The “polypeptide” is also intended to the product modified after the expression for referring to polypeptide, including but not limited to, glycosylation, acetylation, phosphorylation, amidation, by known protection/end-capping group derivatization, proteolytic cleavage or pass through non-naturally occurring amino. The term “polypeptide” also encompasses a naturally-occurring as well as artificial (e.g., engineered or variant) full-length protein as well as a functional fragment of the protein.

As used herein, the term “functional fragment” refers to a portion of a polypeptide/protein that retains some or all of the activity or function (e.g., biological activity or function, such as enzymatic activity or antigen-binding function) of the full-length polypeptide/protein, such as, e.g., the ability to bind to a specific epitope. The functional fragment can be any size, provided that the fragment retains the activity/functionality of the full-length protein.

### Antibody

The term “antibody” is used herein in the broadest sense and encompasses various antibody structures, including but not limited to monoclonal antibodies, polyclonal antibodies, chimeric, humanized antibodies, multi-specific antibodies (e.g., bispecific antibodies), and antigen-binding fragments thereof and similar antigen-binding molecules so long as thy exhibit the desired antigen-binding activity. The “antibody” as used herein can be any type (IgG, IgM, IgD, IgE, IgA and IgY) or the immunoglobulin molecules of classification (IgG1, IgG2, IgG3, IgG4, IgA1 and IgA2) or sub-class according to immunoglobulin (Ig) of the present invention.

As used herein, the terms “Tau11i antibody”, “Tau11i-specific antibody”, “anti-Tau11i antibody”, and “an antibody that specifically recognises Tau11i” refer to an antibody that is capable of binding Tau11i with sufficient affinity and specificity to be useful as a diagnostic and/or therapeutic agent in targeting Tau11i. In some embodiments, the extent of binding of an anti-Tau11i antibody to an unrelated, non-Tau11i protein is less than about 10% of the binding of the antibody to Tau11i as measured by known methods in the art. In certain embodiments, an antibody that binds to Tau11i has a dissociation constant (K_{D}) of ≤1 UM, ≤100 nM, ≤10 nM, ≤1 nM, ≤0.1 nM, ≤0.01 nM, or ≤0.001 nM. The term “Tau11i-specific antibody”, “anti-Tau11i antibody” and “antibody that specifically recognises Tau11i” as used herein, refers to an antibody that binds monomeric Tau11i, oligomeric Tau11i, and/or phosphorylated Tau11i, unless specifically indicated otherwise.

The term “chimeric antibody,” as used herein, refers to at least one antibody molecule in which the amino acid sequence in the constant regions has been altered so that the antibody more closely resembles a human antibody, and still retains its original binding ability.

The term “humanized antibody”, as used herein, refers to at least one antibody molecule in which the amino acid sequence within the variable and constant regions has been altered so that the antibody more closely resembles a human antibody, and still retains its original binding ability.

As used herein, an “antigen-binding fragment” or “antibody fragment” of an antibody refers to an incomplete or isolated portion of the full sequence of the antibody which retains the antigen binding function of the parent antibody. Examples of antibody fragments include but are not limited to Fv, Fab, Fab′, Fab′-SH, F(ab′)2; diabodies; linear antibodies; single-chain antibody molecules (e.g. scFv); and multi-specific antibodies formed from antibody fragments. Fragments of the 8A12 antibody are encompassed by the invention so long as they retain the desired affinity of the full-length antibody. In particular, it may be shorter by at least one amino acid.

As used herein, an “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more.

The term “isolated” is herein defined as a biological component (such as a nucleic acid, peptide or protein) that has been substantially separated, produced apart from, or purified away from other biological components in the cell of the organism in which the component naturally occurs, i.e., other chromosomal and extra-chromosomal DNA and RNA, and proteins. Nucleic acids, peptides and proteins which have been isolated thus include nucleic acids and proteins purified by standard purification methods. The term also embraces nucleic acids, peptides and proteins prepared by recombinant expression in a host cell as well as chemically synthesized nucleic acids.

A description of exemplary, non-limiting embodiments of the invention follows.

The present disclosure is based, in part, on the inventors' discovery that the retention of intron 11 in the Tau gene introduces a premature stop codon, which results in the production of truncated Tau11i polypeptide. In this regard, the inventors have successfully identified that this new C-terminal truncated Tau-11i species not only exhibits biochemical properties that resemble pathological Tau, it is also more abundantly found in AD brain tissues tested. Without being bound by theory, it is believed that the dysregulation of RNA splicing and intron retention is a hallmark of AD pathogenesis, suggesting that Tau11i might be downstream of these events. In light of the above, the polypeptides disclosed herein therefore may advantageously serve as a biomarker and a useful diagnostic for AD.

To this end, provided in one aspect of the present disclosure is an isolated or recombinant polypeptide comprising the following amino acid sequence:
(1) the amino acid sequence HKPGSPVEGEGWDGRVQGV, as set forth in SEQ ID NO: 14 or antigenic fragment thereof, or
(2) the amino acid sequence SPVEGEGWDGRVQG, as set forth in SEQ ID NO: 15, or
(3) an amino acid sequence resulting from substitution, deletion, addition or insertion of one or two or more amino acids in the amino acid sequence set forth in SEQ ID NO: 14 or SEQ ID NO: 15.

In some embodiments, the isolated or recombinant Tau11i-derived polypeptide may consist of amino acid sequence set forth in (1) or (2) with a cysteine residue conjugated to an end. In this regard, the addition of terminal cysteine to the polypeptide sequence may allow its conjugation to carrier proteins, which helps in antibody production. In some embodiments, the isolated or recombinant Tau11i-derived polypeptide may consist of the amino acid sequence HKPGSPVEGEGWDGRVQGV-C set forth in SEQ ID NO: 1 or C-SPVEGEGWDGRVQG set forth in SEQ ID NO: 2.

In some embodiments, the amino acid sequence of the isolated or recombinant polypeptide may comprise or consist of an amino acid sequence resulting from substitution, deletion, addition or insertion of one or two or more amino acids in the amino acid sequence set forth in SEQ ID NO: 1, SEQ ID NO: 2 or SEQ ID NO: 13. In some embodiments, the amino acid sequence may consist of SEQ ID NO: 13.

As those skilled in the art would appreciate, the amino acid sequences of the polypeptides disclosed herein may be sufficiently varied so long as the polypeptides maintain their functionality and can exhibit the required activity (for example, the polypeptide serving as a binding epitope for Tau11i-specific antibodies and/or aptamers).

TABLE 1
Amino acid sequences of polypeptides of the disclosure and nucleotide
sequences of primers used in the present invention

| | |
|---|---|
| Peptide used for | HKPGSPVEGE GWDGRVQGVC |
| generating Tau11i-A | |
| antibody (with | |
| conjugated cysteine) | |
| Amino acid sequence | |
| (Length: 20 amino | |
| acids) | |
| SEQ ID NO: 1 | |
| Peptide used for | CSPVEGEGWD GRVQG |
| generating Tau11i-B | |
| antibody (with | |
| conjugated cysteine) | |
| Amino acid sequence | |
| (Length: 15 amino | |
| acids) | |
| SEQ ID NO: 2 | |
| Xbal-Tau-F | CTATCTAGAG CTGAGCCCCG CCA |
| nucleotide sequence | |
| (Length: 23 bases) | |
| SEQ ID NO: 3 | |
| Sall-Tau-R | AACGTCGACT CACAAACCCT GCTTG |
| Nucleotide sequence | |
| (Length: 25 bases) | |
| SEQ ID NO: 4 | |
| Tau-11e-F | GTGCAAATAG TCTACAAAC |
| Nucleotide sequence | |
| (Length: 19 bases) | |
| SEQ ID NO: 5 | |
| Sall-Tau-11i-R | GTCGACTCAC CAGGACTCCT CC |
| Nucleotide sequence | |
| (Length: 22 bases) | |
| SEQ ID NO: 6 | |
| Tau ex-in11-F | ACCAGGTAGC CCTGTGGAAG |
| Nucleotide sequence | |
| (Length: 20 bases) | |
| SEQ ID NO: 7 | |
| Tau ex-in11-R | CCGCAAGTTT CACACTCAAC |
| Nucleotide sequence | |
| (Length: 20 bases) | |
| SEQ ID NO: 8 | |
| Tau ex-1-F | AAGACCAAGA GGGTGACACG |
| Nucleotide sequence | |
| (Length: 20 bases) | |
| SEQ ID NO: 9 | |
| Tau ex-2-R | ACATCTTCCG CTGTTGGAGT |
| Nucleotide sequence | |
| (Length: 20 bases) | |
| SEQ ID NO: 10 | |
| Gapdh-F | CAATGACCCC TTCATTGACC |
| Nucleotide sequence | |
| (Length: 20 bases) | |
| SEQ ID NO: 11 | |
| Gapdh-R | GACAAGCTTC CCGTTCTCAG |
| Nucleotide sequence | |
| (Length: 20 bases) | |
| SEQ ID NO: 12 | |
| Amino acids encoded | SPVEGEGWDG RVQGVEESW |
| by intron 11 | |
| Amino acid sequence | |
| (Length: 19 amino | |
| acids) | |
| SEQ ID NO: 13 | |
| Peptide used for | HKPGSPVEGE GWDGRVQGV |
| generating Tau11i-A | |
| antibody | |
| Amino acid sequence | |
| (Length: 19 amino | |
| acids) | |
| SEQ ID NO: 14 | |
| Peptide used for | SPVEGEGWDG RVQG |
| generating Tau11i-B | |
| antibody | |
| Amino acid sequence | |
| (Length: 14 amino | |
| acids) | |
| SEQ ID NO: 15 | |
| Tau441 isoform | MAEPRQEFEV MEDHAGTYGL GDRKDQGGYT |
| Amino acid sequence | MHQDQEGDTD AGLKESPLQT PTEDGSEEPG |
| (length: 441 amino | SETSDAKSTP TAEDVTAPLV DEGAPGKQAA |
| acids) | AQPHTEIPEG TTAEEAGIGD TPSLEDEAAG |
| SEQ ID NO: 16 | HVTQARMVSK SKDGTGSDDK KAKGADGKTK |
| | IATPRGAAPP GQKGQANATR IPAKTPPAPK |
| | TPPSSGEPPK SGDRSGYSSP GSPGTPGSRS |
| | RTPSLPTPPT REPKKVAVVR TPPKSPSSAK |
| | SRLQTAPVPM PDLKNVKSKI GSTENLKHQP |
| | GGGKVQIINK KLDLSNVQSK CGSKDNIKHV |
| | PGGGSVQIVY KPVDLSKVTS KCGSLGNIHH |
| | KPGGGQVEVK SEKLDFKDRV QSKIGSLDNI |
| | THVPGGGNKK IETHKLTFRE NAKAKTDHGA |
| | EIVYKSPVVS GDTSPRHLSN VSSTGSIDMV |
| | DSPQLATLAD EVSASLAKQG |
| | L |
| Tau11i isoform | MAEPRQEFEV MEDHAGTYGL GDRKDQGGYT |
| Amino acid sequence | MHQDQEGDTD AGLKESPLQT PTEDGSEEPG |
| (length: 352 amino | SETSDAKSTP TAEDVTAPLV DEGAPGKQAA |
| acids) | AQPHTEIPEG TTAEEAGIGD TPSLEDEAAG |
| SEQ ID NO: 17 | HVTQARMVSK SKDGTGSDDK KAKGADGKTK |
| | IATPRGAAPP GQKGQANATR IPAKTPPAPK |
| | TPPSSGEPPK SGDRSGYSSP GSPGTPGSRS |
| | RTPSLPTPPT REPKKVAVVR TPPKSPSSAK |
| | SRLQTAPVPM PDLKNVKSKI GSTENLKHQP |
| | GGGKVQIINK KLDLSNVQSK CGSKDNIKHV |
| | PGGGSVQIVY KPVDLSKVTS KCGSLGNIHH |
| | KPGSPVEGEG WDGRVQGVEE SW |
| | (underlined region represents amino acids |
| | encoded by intron 11) |
| Sequence of Tau Exon | G TGCAAATAGT CTACAAACCA GTTGACCTGA |
| 11 + Intron 11 + Exon | GCAAGGTGAC CTCCAAGTGT GGCTCATTAG |
| 12 (position 119690 to | GCAACATCCA |
| 124177 of reference | TCATAAACCA Ggtagccctg tggaaggtga gggttgggac |
| sequence | gggagggtgc agggggtgga ggagtcctgg tgaggctgga actgctccag |
| NG_007398.2 | acttcagaag gggctggaaa ggatatttta |
| obtained from NCBI) | ggtagaccta catcaaggaa agtgttgagt gtgaaacttg cgggagccca |
| DNA Sequence | ggaggcgtgg tggctccagc tcgctcctgc ccaggccatg ctgcccaaga |
| (length: 4488 bases) | caaggtgagg cgggagtgaa |
| SEQ ID NO: 18 | gtgaaataag gcaggcacag aaagaaagca catattctcg gccgggcgct |
| | gtggctcacg cctgtaattc cagcactttg ggaggccaag gtgggtggat |
| | catgaggtca ggagattgag |
| | accatcctgg ctaacacagt gaaaccccgt ctctactaaa aatacaaaaa |
| | attagccggg cgtggtggtg ggcgcctgta gtcccagcta ctccggaggc |
| | tgaggcagga aaatggcgtg |
| | aacccggaag gcggagcttg cagtgagcgg agtgagcaga gatcgcgcca |
| | ctgcactcca gcctgggcga cagagcgaga ctccgtctca aaaaaaaaaa |
| | gcacatgttc tcgcttcttt |
| | gtgggatcca ggagatagag aatagaagga tggttaccag aggctgggaa |
| | gggtagtgag gggatggtgg ggggatggtc aatgggtaca aaaaaaatag |
| | aataagacct agtatttgat |
| | agtgcaacag ggtgactata gtcaataata atttaattgt acatttaaaa |
| | ataactaaaa gatagccggg tgcagtggct tacgtctgta atcccagtac |
| | tttgggaggc tgaggtgggc |
| | gtttgagacc agcctggcca acatggtgaa accccatctc tactaaaaat |
| | acaaaaatta gccaggcatg gtggcgggcg cctgtaatcc cagctactcg |
| | ggaggctgag gcaggagaat |
| | cacttgaacc tgggaggcag aggttgcagt gagccgagat cttgccactg |
| | cactccagcc tgggtgacag tgaaactccg tctcaaaaat aaaaataaaa |
| | atacagctgg gcacggtggc |
| | tcacgcctgt aatcccagca ctttgggagg ccgaggcgag cggatcacaa |
| | ggtcaggaga tatagaccat cctggctaac acggtgaaac ccggtctcta |
| | ctaaaaatac aaaaaattag |
| | ccaggcgtgg tggcaggtgc ctatagtccc agctactcac aaggctgagg |
| | caggagaatg gcatgaacct gggaggcgga gcttgcagtg agccgagatt |
| | gtgccactgc actccagcct |
| | gggcgagaga gtgagactcc gtctcaaaac aaaaacaaaa |
| | acaaaaacaa aaacaaacac acaacaaaaa cctaaaagaa |
| | tataaatgga ttgtttgtaa cacaaaggac aaatgtttga |
| | ggggatggat accccatttt ccatgatgtg attattatac attgtgtgtc |
| | tgtatcaaaa catctcatga gccccataaa tatatacacc taactatgta |
| | cccacaaaaa ttaaaaaaat |
| | atatttttta aggtgaagag ggaggcgaga tgctggcctt aacccctaac |
| | ccgttgttct ccctgcaagc tgtccacagg gcctctcaga ctcgaggttc |
| | agctatatgg atgcatgagc |
| | ttggtcccca gccaacatgg gagacacttc accatcggca gcagctacag |
| | cacaggaacc ctgggtcact gccatgtccc ctctgtgact ttgtttaaac |
| | agaaaatgat gctctgggcc |
| | ggctgtggtg gcccacacct ataatcccag caccttggga ggcgggggtg |
| | ggcagattgc ctgaggtcag gagttggaga tcagcctggc cgacatggcg |
| | aaaccccatg tctactaaaa |
| | atacaaaaac tagccaggca tggtggcaca tgcctgtaat cccagctact |
| | tgggaggctg aagcaggaga atcacttgaa cccaggaggc agaggctgag |
| | tgagccaaga tcgtgccaat |
| | gcactccagc ttgggtgagg gagtgagact ccgtctcaaa aaaaaaaaaa |
| | aagaaagaaa aagaaaagaa agtgatccta ctggaaccat gcttactccc |
| | ctccccacct cacactgtgt |
| | agaaattagt gctgtcggcc aggcgcggtg gctcatgcct gtaatcgcag |
| | cactttggga ggccaaggca ggoggatcac gaggtcagga gatcaagacc |
| | atcctggcta acacagtgaa |
| | accctgtctc tactaaaaat acaaaaaatt agccgggcat ggtggcaggc |
| | acctgtagtc ccaactactt gggaggctga ggcaggagaa tggcatgaac |
| | ctgggaggcg gagcttgcag |
| | tgagccaaga tcgcgccact gcataccagc ctaggtgaca gagtgagact |
| | cagcaaaaaa agaaagaaag aaagaaagaa atcagtgctg tctatacttc |
| | tttctgcagt gatggaaata |
| | ttctgtatct gtgctgtcca gtatagtagc cactagctac atgtggcact |
| | tgaaacatgg ctggtacagt tgaggaagag tggctgccat atcggacgac |
| | acagctatag attctgtcac |
| | cccaccccga gagtccagag cggggacttc tgccttaggc cctattcagg |
| | gctgattttt acttgaaccc ttactgtggg aagagaaggc catgagaagt |
| | tcagtctaga atgtgactcc |
| | ttattttctg gctcccttgg acactttgtg ggatttagtc tccctgtgga |
| | aagtattcca caagtggtgc caccacccca gctgtgagag cagctgggag |
| | ctgcttttgt catctttccc |
| | tggaaagtcc tgtgggctgt ctcttcctca tgccttgtcc catgcttggg |
| | catggtgtca agcgtcagga gggagaaagg gtccttattt atttatttag |
| | agagggaccc ttcttctgtt |
| | cccaggctgg agtgcagtgg tgcgatctcg gctcactgca acctccgcct |
| | cctgggttca agtgattctc ctgcctcagc ctcctgagta gctgagatta |
| | caggcacatg ccaacatgcc |
| | tggctaattt tttttttttt tttttttttt tttttttgag atggagttgt actctcattg |
| | cccaggctgg aatgtaatgg cacaatctcg gctcactgca acctccacct |
| | cctggattca |
| | agcaattctc ctgtctcagc ttcccaagta gctgggatta caggtgcccg |
| | ccaccatgct caactaattt ttgtattttt tttttagtag agacgaggtt |
| | tcaccatgtt ggtcagactg |
| | gtctcaaact cctgacctca ggtgatccac ctgcctcggc ctcccaaagt |
| | gctaggatta caggcatgag ccaccacgcc cggcctgaaa gggttcttat |
| | ttagtgtgca ttttgacatt |
| | caatttaatt ccaaggtctt gtggggtcat ggtttacagg atgttgatat |
| | agaaaagact tcacttaatg ggccgggcgc agtggctcat gcctgtaatc |
| | ccagcacttt gggaggccga |
| | ggcaggcaga tcaggaggtc aggagattga gaccatcctg gctaacacag |
| | tgaaacccca tctctactga aaatacaaaa aattagctgg gcgtggtggc |
| | aggcacctgt agtcccagcc |
| | actcggttgg ctgaggcagg agaatggcat gaacccggga ggcggagctt |
| | gcagtgagca gagaccatgc cactgcactc cagcctgggc gacagagcaa |
| | gactctgtct caagaaaaaa |
| | aaaaaaaaaa cagactttac ttactggaag ccaaccaatg tatatttaga |
| | gtaatttttc ctgggctgag ctgtcattta cttttgcagt atctcaagaa |
| | gaagagttta cagtgtaaat |
| | atttgatgca cactttgatt atatagatga agcaaactat tttcaagagc |
| | tttgcaagga cttacttgta tccaaacacc attctaaagg agtcttacct |
| | acttctaaag gctggtctct |
| | acttggaacc acttgcttgg ccctggttca agtcctgctg caaacctgga |
| | agtcctgtca ttgtcttctt ccctccagag cagtggcacc caatctaatt |
| | tttgctgtgc cccagcagcc |
| | cctggcactt tgccctgtag actgcagacc tcatgtaatg tatgttaagt |
| | ccacagaacc acagaagatg atggcaagat gctcttgtgt gtgttgtgtt |
| | ctagGAGGTG GCCAGGTGGA |
| | AGTAAAATCT GAGAAGCTTG ACTTCAAGGA |
| | CAGAGTCCAG TCGAAGATTG GGTCCCTGGA |
| | CAATATCACC CACGTCCCTG GCGGAGGAAA |
| | TAAAAAG |
| | DNA sequence of exon 11 is represented by the region that |
| | is underlined and in uppercase. |
| | DNA sequence of intron 11 is represented by the DNA |
| | sequence in lowercase. Bold and underlined sequence “tga” |
| | represents the premature stop codon in intron 11. |
| | DNA sequence of exon 12 is represented by the region that |
| | is bold, underlined, italicised and in uppercase. |
| Tau E10-11 Fw | GATTGGGTCC CTGGACAATA |
| Nucleotide sequence | |
| (Length: 20 bases) | |
| SEQ ID NO: 19 | |
| Tau E10-11 Rv | GACACCACTG GCGACTTGTA |
| Nucleotide sequence | |
| (Length: 20 bases) | |
| SEQ ID NO: 20 | |
| TauE2i Fwd | ACTCCAACAG CGGAAGGTGG |
| Nucleotide sequence | |
| (Length: 20 bases) | |
| SEQ ID NO: 21 | |
| TauE2i Rv | CAAAGCACCG AAGGAGTGAG |
| Nucleotide sequence | |
| (Length: 20 bases) | |
| SEQ ID NO: 22 | |
| HBB E1E2 Fw | ACGTGGATGA AGTTGGTGGT |
| Nucleotide sequence | |
| (Length: 20 bases) | |
| SEQ ID NO: 23 | |
| HBB E1E2 Rv | CAGCATCAGG AGTGGACAGA |
| Nucleotide sequence | |
| (Length: 20 bases) | |
| SEQ ID NO: 24 | |
| HBB E1-i1 Fw | CAAGGTGAAC GTGGATGAAG |
| Nucleotide sequence | |
| (Length: 20 bases) | |
| SEQ ID NO: 25 | |
| HBB E1-i1 Rv | GTCTCCACAT GCCCAGTTTC |
| Nucleotide sequence | |
| (Length: 20 bases) | |
| SEQ ID NO: 26 | |
| HDAC2 E2E3 Fw | GACAGGGTCA TCCCATGAAG |
| Nucleotide sequence | |
| (Length: 20 bases) | |
| SEQ ID NO: 27 | |
| HDAC2 E2E3 Rv | TTGTCATTTC TTCGGCAGTG |
| Nucleotide sequence | |
| (Length: 20 bases) | |
| SEQ ID NO: 28 | |
| Heavy Chain Variable | MGRLTSSFLL LIVPAYVLSQ VTLKESGPGI LQPSQTLSLT |
| Region of mAb 8A12 | CSFSGFSLST SGMGVGWIRQ PSGKGLEWLA |
| Amino Acid Sequence | HIWWDDVKRY |
| (Length: 136 amino | NPALKSRLTI SKDTSSSQVF LKIASVDTAD TATYYCVRTI |
| acids) | LWMDYWGQGT SVTVSS |
| SEQ ID NO: 29 | |
| Heavy Chain Variable | ATGGGCAGGC TTACTTCTTC ATTCCTGCTA |
| Region | CTGATTGTCC |
| DNA Sequence | CTGCATATGT CCTGTCCCAG GTTACTCTGA |
| (Length: 408 bp) | AAGAGTCTGG |
| SEQ ID NO: 30 | CCCTGGGATA TTGCAGCCCT CCCAGACCCT |
| | CAGTCTGACT |
| | TGTTCTTTCT CTGGGTTTTC ACTGAGCACT |
| | TCTGGTATGG |
| | GTGTAGGCTG GATTCGTCAG CCATCAGGGA |
| | AGGGTCTGGA |
| | GTGGCTGGCA CACATTTGGT GGGATGATGT |
| | CAAGCGCTAT |
| | AACCCAGCCC TGAAGAGCCG ACTGACTATC |
| | TCCAAGGATA |
| | CCTCCAGCAG CCAGGTATTC CTCAAGATCG |
| | CCAGTGTGGA |
| | CACTGCAGAT ACTGCCACAT ATTACTGTGT |
| | TCGAACCATT |
| | CTATGGATGG ACTACTGGGG TCAAGGAACC |
| | TCAGTCACCG |
| | TCTCCTCA |
| HCDR1 | TSGMGVG |
| Amino Acid Sequence | |
| (Length: 7 amino | |
| acids) | |
| SEQ ID NO: 31 | |
| HCDR2 | HIWWDDVKRY NPALKS |
| Amino Acid Sequence | |
| (Length: 16 amino | |
| acids) | |
| SEQ ID NO: 32 | |
| HCDR3 | TILWMDY |
| Amino Acid Sequence | |
| (Length: 7 amino | |
| acids) | |
| SEQ ID NO: 33 | |
| Light Chain Variable | MDSQAQVLIL LLLWVSGTCG DIVMSQSPSS |
| Region of mAb 8A12 | LAVSAGEKVT |
| Amino Acid Sequence | MSCKSSQSLL NSRTRKNYLA WYQQKPGQSP |
| (Length: 132 amino | KLLIYWASTR |
| acids) | ESGVPDRFTG SGSGTDFTLT ISSVQAEDLA |
| SEQ ID NO: 34 | VYYCKQSYNL |
| | FTFGSGTKLE IK |
| Light Chain Variable | ATGGATTCAC AGGCCCAGGT TCTTATATTG |
| Region | CTGCTGCTAT |
| DNA Sequence | GGGTATCTGG TACCTGTGGG GACATTGTGA |
| (Length: 396 bp) | TGTCACAGTC |
| SEQ ID NO: 35 | TCCATCCTCC CTGGCTGTGT CAGCAGGAGA |
| | GAAGGTCACT |
| | ATGAGCTGCA AATCCAGTCA GAGTCTGCTC |
| | AACAGTAGAA |
| | CCCGAAAGAA CTACTTGGCT TGGTACCAGC |
| | AGAAACCAGG |
| | GCAGTCTCCT AAACTGTTGA TCTACTGGGC |
| | ATCCACTAGG |
| | GAATCTGGGG TCCCTGATCG CTTCACAGGC |
| | AGTGGATCTG |
| | GGACAGATTT CACTCTCACC ATCAGCAGTG |
| | TGCAGGCTGA |
| | AGACCTGGCA GTTTATTACT GCAAGCAATC |
| | TTATAATCTA |
| | TTCACGTTCG GCTCGGGGAC AAAGTTGGAA ATAAAA |
| LCDR1 | KSSQSLLNSR TRKNYLA |
| Amino Acid Sequence | |
| (Length: 17 amino | |
| acids) | |
| SEQ ID NO: 36 | |
| LCDR2 | WASTRES |
| Amino Acid Sequence | |
| (Length: 7 amino | |
| acids) | |
| SEQ ID NO: 37 | |
| LCDR3 | KQSYNLFT |
| Amino Acid Sequence | |
| (Length: 8 amino | |
| acids) | |
| SEQ ID NO: 38 | |

It would be appreciated that the polypeptides of the present invention can be produced according to a known peptide synthesis method, for example, solid phase synthesis process, liquid phase synthesis process and the like. The obtained polypeptides can be purified and isolated by a known purification method, for example, solvent extraction, distillation, column chromatography, liquid chromatography, recrystallization, a combination of these, and the like. The polypeptides of the present invention can also be produced by culturing a transformant containing the nucleic acids encoding the polypeptides and separating and purifying the polypeptides from the resulting culture. The polypeptides disclosed herein are suitable for or can be used for preparing epitope-specific antibodies, mapping antibody epitopes and enzyme binding sites and designing novel enzymes, drugs and vaccines.

Accordingly in another aspect, there is provided an antibody or antigen-binding fragment thereof capable of binding to the polypeptides of the present disclosure.

Preferably, the antibody or the antigen-binding fragment of the present invention is capable of binding to Tau11i-specific epitopes with sufficient affinity and specificity such that the antibody or the antigen-binding fragment thereof is useful as a diagnostic and/or therapeutic agent in targeting Tau11i. In some embodiments, the antibody described herein may be a polyclonal antibody or a monoclonal antibody.

In some embodiments, the isolated antibody or antigen-binding fragment thereof specifically binds to the polypeptide comprising the amino acid sequence HKPGSPVEGEGWDGRVQGV (SEQ ID NO: 14) or antigenic fragment thereof, or the amino acid sequence SPVEGEGWDGRVQG, (SEQ ID NO: 15), or an amino acid sequence resulting from substitution, deletion, addition or insertion of one or two or more amino acids in the amino acid sequence set forth in SEQ ID NO: 14 or SEQ ID NO: 15.

In some embodiments, the isolated antibody or antigen-binding fragment thereof specifically binds to the polypeptide consisting of the amino acid sequence set forth in SEQ ID NO:1 or SEQ ID NO: 2.

In some embodiments, the isolated antibody or antigen-binding fragment thereof specifically binds to the polypeptide consisting of an amino acid sequence resulting from substitution, deletion, addition or insertion of one or two or more amino acids in the amino acid sequence set forth in SEQ ID NO: 1, SEQ ID NO: 2 or SEQ ID NO: 13.

In some embodiments, the antibody or antigen-binding fragment thereof comprises a heavy chain variable region comprising complementarity determining regions HCDR1 having an amino acid sequence set forth in SEQ ID NO: 31, HCDR2 having an amino acid sequence set forth in SEQ ID NO: 32, and HCDR3 having an amino acid sequence set forth in SEQ ID NO: 33, and/or a light chain variable region comprising complementarity determining regions LCDR1 having an amino acid sequence set forth in SEQ ID NO: 36, LCDR2 having an amino acid sequence set forth in SEQ ID NO:37, and LCDR3 having an amino acid sequence set forth in SEQ ID NO: 38. In some embodiments, the antibody or antigen-binding fragment thereof comprises a light chain variable region comprising the amino acid sequence shown as residues 1 to 132 of SEQ ID NO: 34 and a heavy chain variable region comprising the amino acid sequence shown as residues 1 to 136 of SEQ ID NO: 29.

In a further aspect, there is provided a method for preparing a Tau11i-specific antibody using the polypeptides disclosed herein, the method comprising
(1) injecting the polypeptide of the present disclosure into an animal to induce an immune response to the polypeptide;
(2) isolating antibodies that bind to the polypeptide;
(3) selecting antibodies of (2) that bind to Tau11i but do not bind to full length Tau thereby producing a Tau11i-specific antibody.

In some embodiments, the method may comprise (1) injecting a non-human mammal with a Tau11i-derived polypeptide of the present disclosure to form a B cell specific for Tau11i; (2) selecting a B cell specific to the Tau11i; (3) fusing the selected B cell with an immortalized cell to produce a hybridoma, wherein the hybridoma is capable of producing an antibody or a fragment thereof specific for the Tau11i; and (4) optionally isolating the antibody from the hybridoma and sequencing the variable heavy and light chains.

In some embodiments, the non-human mammal may be a mouse, rabbit, cow, goat or sheep.

In a further aspect, there is provided a polynucleic acid molecule encoding the polypeptide, the antibody or antigen binding fragment thereof as disclosed herein. In some embodiments, the polynucleic acid may be a cDNA.

Also provided in another aspect are oligonucleotides for amplifying a polynucleotide sequence spanning the junction of exon 11 and intron 11 of a Tau gene. In some embodiments, the oligonucleotides consist of the nucleotide sequences set forth in SEQ ID NO: 7 and/or SEQ ID NO: 8.

In another aspect, there is provided a method for detecting the polypeptide of the present disclosure, the method comprises: a step of contacting a sample suspected of comprising the polypeptide disclosed herein with the antibody or antigen-binding fragment of the present disclosure. For the purposes of the present invention, the method is preferably in vitro.

In another aspect, there is also provided a method for diagnosing Alzheimer's disease or identifying a subject at risk of developing Alzheimer's disease, wherein the method comprises: contacting a sample derived from the subject with the antibody or antigen binding fragment thereof as described herein.

In this regard, it would be appreciated that the antibody or antigen-binding fragment of the present disclosure may bind specifically to the polypeptides disclosed herein to form an antibody-polypeptide complex. Accordingly, the methods disclosed herein may further comprise the step of detecting the antibody-polypeptide complex, wherein the presence of said antibody-polypeptide complex may be used to inform the diagnosis and/or assessment of the risk of said subject in developing AD.

Also provided in a further aspect, is a method for diagnosing Alzheimer's disease or identifying a subject at risk of developing Alzheimer's disease, wherein the method comprises: conducting a nucleic acid detection assay to assess the abundance of intron 11 retaining mRNA in a sample from the subject.

In some embodiments, the nucleic acid detection assay may comprise amplifying a polynucleotide sequence spanning the junction of exon 11 and intron 11 of a Tau gene. In various embodiments, the amplification involves using oligonucleotides having the nucleotide sequences set forth in SEQ ID NOs: 7 and/or 8.

In this regard, it would be appreciated that the abundance of intron 11 retaining mRNA in said sample may be compared with the abundance in normal controls to establish the diagnosis or risk factor of the subject in developing AD. As described herein, the presence of Tau11i polypeptides are typically significantly higher in AD patients. Thus, it would be appreciated that a standard value for normals with appropriate error bars may be generated by averaging the results from a statistically meaningful sample of individuals known not to be afflicted by this condition. Accordingly, determining and/or analyzing the degree of difference in abundance of intron 11 retaining mRNA in said sample against a control may thus be used to inform the diagnosis and/or assessment of the risk of said subject in developing AD.

In some embodiments, the sample may be a biological fluid or tissue. For example, a biological fluid may include but not limited to, blood and fractions thereof such as plasma or serum, cerebrospinal fluid, urine, lymphatic fluids, synovial fluid, pericardial fluid, peritoneal fluid, amniotic fluid, saliva, nasal fluid etc.

In some embodiments, the sample may be brain tissue, cerebrospinal fluid, blood plasma, saliva, lacrimal fluid or urine.

Provided in a further aspect is a method for screening a candidate therapeutic or prophylactic drug for Alzheimer's disease, comprising using any of the following as an index: (1) reduction of the amount of Tau protein in the body of an Alzheimer's disease subject or a subject at risk of developing Alzheimer's disease, or (2) inhibition of production of Tau protein in the body of an Alzheimer's disease subject or a subject at risk of developing Alzheimer's disease; wherein the Tau protein comprises the amino acid sequence of the polypeptide as disclosed herein.

In another aspect of the present disclosure there is also provided a kit comprising the antibody or antigen-binding fragment of the present invention. In some embodiments, the kit may further comprise the polypeptide of the present disclosure.

For the purposes of the present invention, the kit as herein described is preferably equipped to conduct the one or more methods as described above. In some embodiments, the kit may comprise a reagent to detect the antibody or the antigen-binding fragment of the present invention. In other embodiments, the kit may comprise a reagent to detect a complex that may have been formed between the antibody or the antigen-binding fragment thereof with a test sample. Reagents that are suitable for detecting the complex may include reagents that may incorporate a detectable label, such as a fluorophore, radioactive moiety, enzyme, biotin/avidin label, chromophore, chemiluminescent label, or the like.

The kits may also optionally include other reagents required to conduct a diagnostic or prognostic assay as described herein, such as buffers, salts, enzymes, enzyme co-factors, substrates, detection reagents, and the like. Other components, such as buffers and solutions for the isolation and/or treatment of a test sample (e.g. pretreatment reagents), may also be included in the kit. The kit may additionally include one or more other controls. One or more of the components of the kit may be lyophilized and the kit may further comprise reagents suitable for the reconstitution of the lyophilized components.

Apart from the antibodies or antigen binding fragments disclosed herein, it is also envisaged that aptamers may be suitably adapted for use in the practice of the present invention. Aptamers are nucleic acid probes that form specific three dimensional formations, and are capable of binding to defined targets such as polypeptides with high affinity and specificity. In this regard, it would be appreciated by a skilled artisan that methods for screening epitope-specific aptamers are known in the art, for example, via the systematic evolution of ligands by exponential enrichment (SELEX) process (for example, as described in Teng et al., J. Am. Chem. Soc., 140 (43), 14314-14323 (2018), incorporated herein by reference).

Accordingly, provided herein in another aspect is a method of screening and/or selecting a Tau11i epitope-specific aptamer, the method comprises: (a) conducting systematic evolution of ligands by exponential enrichment (SELEX) technique to identify Tau11i epitope-specific aptamers, wherein the SELEX technique comprises providing a polypeptide comprising the amino acid sequence set forth in any one of SEQ ID NOs. 1, 2, 13, 14 or 15 as a target ligand.

Accordingly, provided in another aspect is an aptamer capable of binding to a polypeptide comprising the amino acid sequence set forth in any one of SEQ ID NOs. 1, 2, 13, 14 or 15. In some embodiments, the aptamer may be a DNA aptamer or RNA aptamer.

In a further aspect, there is also provided a method for diagnosing Alzheimer's disease or identifying a subject at risk of developing Alzheimer's disease, wherein the method comprises: contacting a sample derived from the subject with the Tau11i epitope-specific aptamer as provided herein.

Unless otherwise indicated or otherwise evident from the context and understanding of one of ordinary skill in the art, values that are expressed as ranges can assume any specific value or subrange within the stated ranges in various embodiments, to the tenth of the unit of the lower limit of the range, unless the context clearly dictates otherwise. “About” in reference to a numerical value generally refers to a range of values that fall within ±10%, in some embodiments ±5%, in some embodiments ±1%, in some embodiments ±0.5% of the value unless otherwise stated or otherwise evident from the context. In any embodiment in which a numerical value is prefaced by “about”, an embodiment in which the exact value is recited is provided. Where an embodiment in which a numerical value is not prefaced by “about” is provided, an embodiment in which the value is prefaced by “about” is also provided. Where a range is preceded by “about”, embodiments are provided in which “about” applies to the lower limit and to the upper limit of the range or to either the lower or the upper limit, unless the context clearly dictates otherwise. Where a phrase such as “at least”, “up to”, “no more than”, or similar phrases, precedes a series of numbers, it is to be understood that the phrase applies to each number in the list in various embodiments (it being understood that, depending on the context, 100% of a value, e.g., a value expressed as a percentage, may be an upper limit), unless the context clearly dictates otherwise. For example, “at least 1, 2, or 3” should be understood to mean “at least 1, at least 2, or at least 3” in various embodiments. It will also be understood that any and all reasonable lower limits and upper limits are expressly contemplated.

Having now generally described the invention, the same will be more readily understood through reference to the following examples which are provided by way of illustration, and are not intended to be limiting of the present invention.

### EXAMPLES

Standard molecular biology techniques known in the art and not specifically described were generally followed as described in Green and Sambrook, Molecular Cloning: A Laboratory Manual, Cold Springs Harbor Laboratory, New York (2012).

### Example 1: Materials and General Methods

### High-Throughput Analysis of Intron Retention

DLPFC datasets of healthy control subjects and AD patients (syn21088596) (de Jager et al., Sci. Data, 5 (1), 1-13 (2018)) in the Religious Orders Study/Memory and Aging Project (ROSMAP) were downloaded from AD Knowledge Portal. RNA-sequencing reads were first mapped against a human reference genome (GRCh38.100) and intron retention (IR) was quantified by IRFinder pipeline software using default setting (Middleton et al., Genome Biol. 18, 1-11 (2017)). The IR ratio was calculated by dividing intronic abundance by the sum of intronic and exonic abundance. Intron 11 of the Tau (MAPT) gene (MAPT/ENSG00000186868/clean/17:46014324-46018617: +) investigated in this study has a minimal splicing sequencing depth of greater than five reads. Generalized Linear Model from DESeq2 software (Love et al., Genome Biol. 15 (12), 1-21. (2014)) was applied to measure the differential IR ratio between control subjects and AD patients, where p-value <0.05 was considered significant. A normalized IR ratio from an individual human subject determined from DESeq2 was used to generate a dot plot with “ggplot” function in “ggplot2” package software (https://ggplot2.tidyverse.org). Counts for Tau gene were generated using featureCounts (v.2.0.1.) software and differential expression was calculated by DESeq2 (v.1.30.1) software.

### Cell Lines and Human Brain Tissues

Human NSC (XCL-1, SC-001-1V) were purchased from XCell Science Inc. and differentiated according to manufacturer's protocol. Brain total RNA, protein lysates and frozen tissue sections were purchased from MYBIOSOURCE and BIOCHAIN. Parietal lobe (BA40) samples C6, C7, A6, and A7 were previously described (e.g., Chua, et al., Neurochem. Int. 152, 105251 (2022)).

### Human Brain Samples and Tissue Sections (Donors' Information)

“C” denotes Control and “A” denotes Alzheimer disease.

Total protein lysates from different brain regions were purchased from:

### (a) Mybiosource

1) C1: Adult Normal, Brain, Temporal Lobe Lysate (MBS657128)—male, 77 years old.
2) A1: Adult AD, Brain, Temporal Lobe Lysate (MBS657032)—male, 87 years old
3) C4: Adult Normal, Brain, Frontal Lobe Lysate (MBS657031)—male, 60 years old.
4) A4: Adult AD, Brain, Frontal Lobe Lysate (MBS657343)—female, 85 years old.
5) C5: Adult Normal, Brain, Hippocampus Lysate (MBS657489)—male, 60 years old.
6) A5: Adult AD, Brain, Hippocampus Lysate (MBS657034)—female, 93 years old.

### (b) Biochain

1) C8: Adult Normal, Brain, Temporal Lobe Lysate (P1234078)—female, 70 years old.
2) A1: Adult AD, Brain, Temporal Lobe Lysate (P1236978Alz)—male, 87 years old
3) C9: Adult Normal, Brain, Frontal Lobe Lysate (P1234051)—male, 82 years old.
4) C2: Adult Normal, Brain, Amygdala Lysate (P1234036)—male, 22 years old.
5) A2: Adult AD, Brain, Amygdala Lysate (P1236036Alz)—female, 65 years old.
6) C3: Adult Normal, Brain, Parietal lobe Lysate (P1234066)—male, 66 years old.
7) A3: Adult AD, Brain, Parietal lobe Lysate (P1236066Alz)—male, 73 years old.

The following samples were obtained from Newcastle Brain Tissue Resource, part of the Brains for Dementia Research network.

1) C6: Adult Normal, Brain, Parietal Lobe Lysate (283/96)—male, 77 years old.
2) A6: Adult AD, Brain, Parietal Lobe Lysate (120/09)—female, 85 years old.
3) C7: Adult Normal, Brain, Parietal lobe Lysate (308/09)—male, 66 years old.
4) A7: Adult AD, Brain, Parietal lobe Lysate (350/09)—female, 98 years old.

Frozen tissue sections were purchased from BIOCHAIN (BioChain Institute Inc., CA, USA).
1) C10: Adult Normal, Brain, Temporal Lobe ((T1234078)—male, 26 years old.
2) A10: Adult AD, Brain, Temporal Lobe (T1236078Alz)—male, 73 years old.

Total RNA from different brain regions (AMSBIO) were purchased from BIOCHAIN.
1. AD Brain, R1236035Alz-50 (Lot #: A507294)—male, 87 years old.
2. Normal Brain R1234035-P (Lot #: C404081)-5 males, pooled, 21-29 years old.
3. AD Frontal Lobe, R1236051Alz-10 (Lot #: B204064)—male, 87 years old.
4. Normal Frontal Lobe, R1234051-P (Lot #: B804011)-5 males, pooled, 22-29 years old.
5. AD Temporal Lobe, R1236078Alz-10 (Lot #: B408032)—male, 80 years old.
6. Normal Temporal Lobe, R1234078-P (Lot #: B411025)-5 males, pooled, 23-27 years old.

### Cell Culture

HEK 293T cells (Clontech, 632180) were maintained in Dulbecco's modified Eagle's medium (DMEM) GlutaMAX™ (Gibco) supplemented with 10% FBS, 1×nonessential amino acids (NEAA) and 1.0 mM sodium pyruvate.

Human neural stem cells (NSC) (XCL-1, SC-001-1V, XCell Science Inc.) were seeded on matrigel (Corning, 354277) and cultured in neural expansion medium [1:1 ratio of DMEM GlutaMAX™ to Ham's F-12 Nutrient Mix (both Gibco), 1×NEAA, 1× N-2 supplement (Gibco, 17502048), 1× B-27 supplement minus vitamin A (Gibco, 12587010), 0.5× GlutaMAX (Gibco, 35050061), 20 ng/ml human FGF-basic (Gibco, PHG0261), 2 μg/ml Heparin Sulfate (Sigma, H4784)]. Viral infected NSCs were differentiated according to manufacturer's protocols. Briefly, cells were seeded in NSC maintenance media at a density of 0.04×10⁶ cells/cm² on poly-ornithine (20 μg/mL) & laminin (10 μg/mL)-coated plates (Day 0). Cells were then replenished with neuronal induction media on Day 1, 3 and 5. On Day 6, neuronal precursor cells formed (NPCs) were harvested by StemPro™ Accutase™ (Gibco, A1110501) and replated on poly-ornithine/laminin-coated wells at 0.04×10⁶ cells/cm² in neuronal maturation medium. Neurons were cultured for designated duration with media change every alternate day.

### Transient Transfection and Lentiviral Transduction

Full-length Tau and Tau-11i expression constructs were transiently transfected into HEK 293T cells using Lipofectamine® 3000 reagent (Invitrogen, L3000008) according to manufacturer protocol. Cells were harvested 48 h post-transfection for analyses.

Lenti-X lentiviral expression system (Clontech) was used to generate stable NSC lines that expressed either full-length (FL) Tau or Tau-11i proteins. Briefly, lentivirus was prepared by transfecting 293T cells with a mixture that contained 7 μg of lentiviral plasmids (pLVX-Flag-Tau-FL or pLVX-Flag-Tau-11i) and Lenti-X packaging single shot reagent (VSV-G) (Clontech, 631275). Media harvested 24 h and 72 h post transfection was combined and concentrated using Lenti-X concentrator (Clontech, 631231) to produce lentiviral supernatant. NSCs were infected with lentiviral supernatant in the presence of polybrene (12 μg/ml). After 48 h of viral transduction, stable clones were selected with 0.1 μg/ml of puromycin. Expression of Tau-FL and Tau-11i in stable lines were validated using western blot and immunofluorescence.

### Cloning of Tau Constructs

Full-length (FL) Tau gene was PCR amplified from VN-Tau plasmid (Addgene 87368) using XbaI-Tau-F and SalI-Tau-R primers and blunt-end cloned into pBlueScript II SK+vector at EcoRV site to get PBS-Tau-FL. Following XbaI/SalI restriction of PBS-Tau-FL, full-length Tau fragment was cloned into pLVX-Flag-IRES-puro lentiviral vector at XbaI/PspXI sites to get pLVX-Flag-Tau-FL.

Three steps were taken to make Tau-11i construct. (i) Partial Tau exon and intron 11 region was PCR amplified from human genomic DNA using Tau-Ile-F and SalI-Tau-11i-R primers and cloned into pGEMT vector (Promega) to get pGEMT-Tau-ex/in-11. (ii) BstEII cuts uniquely within exon 11. Fragment of Tau exon and intron 11, released by BstEII/SalI restriction of pGEMT-Tau-ex/in-11, was then used to replace 3′-end of full-length Tau in PBSTau-FL to get PBS-Tau-11i. (iii) Following XbaI/SalI restriction of PBS-Tau-11i, Tau-11i fragment was cloned into pLVX-Flag-IRES-puro vector at XbaI/PspXI sites to get pLVX-Flag-Tau-11i. DNA sequencing was performed at each step to verify sequence integrity.

Xbal-Tau-F
(SEQ ID NO: 3)
5′-CTATCTAGAGCTGAGCCCCGCCA-3′
Sall-Tau-R
(SEQ ID NO: 4)
5′-AACGTCGACTCACAAACCCTGCTTG-3′
Tau-11e-F
(SEQ ID NO: 5)
5′-GTGCAAATAGTCTACAAAC-3′
Sall-Tau-11i-R
(SEQ ID NO: 6)
5′-GTCGACTCACCAGGACTCCTCC-3′

### Preparation of Customized Tau11i Antibodies

Two custom anti-Tau11i polyclonal antibodies were generated by GenScript (GenScript Biotech Corporation, NJ, USA) using peptides HKPGSPVEGEGWDGRVQGV-C(SEQ ID NO: 1) and C-SPVEGEGWDGRVQG (SEQ ID NO: 2) as antigens, where C is a conjugated cysteine.

A custom anti-Tau11i monoclonal antibody, 8A12, was also generated by GenScript using a peptide with the amino acid sequence set forth in SEQ ID NO: 15 as the antigen. The monoclonal antibody 8A12 comprises a heavy chain variable region having the amino acid sequence set forth in SEQ ID NO: 29 and a light chain variable region having the amino acid sequence set forth in SEQ ID NO: 34 (Table 1). The monoclonal antibody 8A12 comprises a heavy chain variable region comprising complementarity determining regions HCDR1 having an amino acid sequence set forth in SEQ ID NO: 31, HCDR2 having an amino acid sequence set forth in SEQ ID NO: 32, and HCDR3 having an amino acid sequence set forth in SEQ ID NO: 33, and a light chain variable region comprising complementarity determining regions LCDR1 having an amino acid sequence set forth in SEQ ID NO: 36, LCDR2 having an amino acid sequence set forth in SEQ ID NO: 37, and LCDR3 having an amino acid sequence set forth in SEQ ID NO: 38 (Table 1).

### Sarkosyl Extraction

Sarkosyl soluble and insoluble fractions were prepared accordingly published protocol (Smith et al., Neuropathol. 46 (7), 641-653 (2020)). Tau expressing 293T cells and NPC stable lines were resuspended with 320 μl per 3e6 cells of low salt buffer (50 mM HEPES pH 7.6, 250 mM sucrose, 1 mM EDTA pH 8.0) with protease inhibitors cocktail (Roche), followed by addition of 40 μl of 5 M NaCl and 40 μl of 10% sarkosyl. Cell lysates were mixed thoroughly by pipetting, incubated on ice for 15 min with agitation, followed by brief sonication (10s ON/30s OFF, high settings, 3 cycles, Bioruptor UCD-200TO). After complete resuspension, 40 μl of cell lysates were harvested as “total” lysate. Following maximum speed centrifugation at 4 °C for 1 h, supernatant was carefully collected as “soluble” extract. The insoluble pellet was gently washed with low salt buffer (with 0.5 M NaCl and 1% sarkosyl) and centrifuged at maximum speed at 4 °C for 10 mins to remove wash buffer. Laemmli buffer was added to the “total” lysate, “soluble” fraction, and “insoluble” pellet, boiled for 10 min, and subjected to immunoblot assay.

### Immunoblot and Antibodies

Different sarkosyl fractions, brain tissues and cellular lysates were boiled in 1× Laemmli buffer, resolved in SDS-PAGE gel and transferred to PVDF membrane by standard western protocol. The antibodies used were anti-Flag (Sigma, F1804), polyclonal anti-Tau11i (GenScript), monoclonal anti-Tau-11i antibody 8A12, anti-Tau46 (CST, 4019S), anti-Tau 4-repeat isoform RD4 (4R, Millipore, 05-804), anti-pTau-S202/T205 (AT8, Invitrogen, MN1020), anti-pTau-T181 (CST, 12885S), anti-pTau-S396 (PHF1, Santa Cruz, sc-32275) and anti-β-actin (Sigma, A2228).

### Immunofluorescence

Immunostaining of neurons and image quantification were performed as previously described (Rao et al., Nucleic Acids Res., 48 (3), 1225-1238 (2020)). Briefly, mature neurons cultured in 12-well plates were fixed with 4% paraformaldehyde in PBS for 20 min, followed by two rounds of PBS washes and 1 h of blocking (10% normal goat serum, 0.1% triton-X 100 in PBS) at room temperature. Neurons were then stained with anti-MAP2 (Millipore, MAB3418), anti-α-tubulin (Santa Cruz, sc-53029) and anti-Flag (Sigma, F1804) antibodies (5 μg/ml in blocking buffer) overnight at 4 °C. After three rounds of PBS washes the following day, neurons were incubated with anti-mouse 594 and anti-rabbit FITC secondary antibodies (1:500, Invitrogen) for 2 h at room temperature. Cells were washed thrice with PBS and mounted in VECTASHIELD with DAPI (Vector labs). Confocal microscope (FV3000, 10× objective) was used to capture at least three fields of images for each condition. The signal threshold was adjusted to obtain optimal coverage of the Flag and α-tubulin staining. The relative fluorescence intensity of FLAG over α-tubulin was quantified using ImageJ software.

Validation of IR by Quantitative PCR (qPCR)

Total RNA of different brain tissues from healthy control subjects and AD patients (Amsbio) were purchased from Biochain. High-capacity cDNA reverse transcription kit (Applied Biosystems) was used to generate cDNA libraries from 1 μg of total RNA pre-treated with DNase I (Thermo Scientific). Real-time quantitative PCR was carried out in triplicates (6 μl/reaction) with 0.2 μM of primers and 2× SYBR green master mix (Thermo Scientific) using 7900HT Fast Real-Time PCR machine (Applied Biosystems). Relative level of Tau-11i (Tau ex-in11-F/R) was calculated using 2^{-ΔCT} method where threshold cycle (CT) values were normalized to either Tau exon1/exon2 (Tau ex-1-F/ex-2-R) or Gapdh expression.

Tau ex-in11-F
(SEQ ID NO: 7)
5′-ACCAGGTAGCCCTGTGGAAG-3′
Tau ex-in11-R
(SEQ ID NO: 8)
5′-CCGCAAGTTTCACACTCAAC-3′
Tau ex-1-F
(SEQ ID NO: 9)
5′-AAGACCAAGAGGGTGACACG-3′
Tau ex-2-R
(SEQ ID NO: 10)
5′-ACATCTTCCGCTGTTGGAGT-3′
Gapdh-F
(SEQ ID NO: 11)
5′-CAATGACCCCTTCATTGACC-3′
Gapdh-R
(SEQ ID NO: 12)
5′-GACAAGCTTCCCGTTCTCAG-3′

### Human Total RNA and Protein Lysates.

Total protein lysates from different brain regions were purchased from MyBiosource, Inc. (San Diego, USA).
1. Adult Normal, Brain, Temporal Lobe Lysate (MBS657128)—male, 77 years old
2. Adult AD, Brain, Temporal Lobe Lysate (MBS657032)—male, 87 years old.
3. Adult Normal, Brain, Frontal Lobe Lysate (MBS657031)—male, 60 years old.
4. Adult AD, Brain, Frontal Lobe Lysate (MBS657343)—female, 85 years old.
5. Adult Normal, Brain, Hippocampus Lysate (MBS657489)—male, 60 years old.
6. Adult AD, Brain, Hippocampus Lysate (MBS657034)—female, 93 years old.

Total RNA from different brain regions (Amsbio) were purchased from Biochain.
1. AD Brain, R1236035Alz-50 (Lot #: A507294)—male, 87 years old.
2. Normal Brain R1234035-P (Lot #: C404081)-5 males, pooled, 21-29 years old
3. AD Frontal Lobe, R1236051Alz-10 (Lot #: B204064)—male, 87 years old.
4. Normal Frontal Lobe, R1234051-P (Lot #: B804011)-5 males, pooled, 22-29 years old.
5. AD Temporal Lobe, R1236078Alz-10 (Lot #: B408032)—male, 80 years old.
6. Normal Temporal Lobe, R1234078-P (Lot #: B411025)-5 males, pooled, 23-27 years old.

### Example 2: Increased Retention of Tau Intron 11 in AD Cohorts

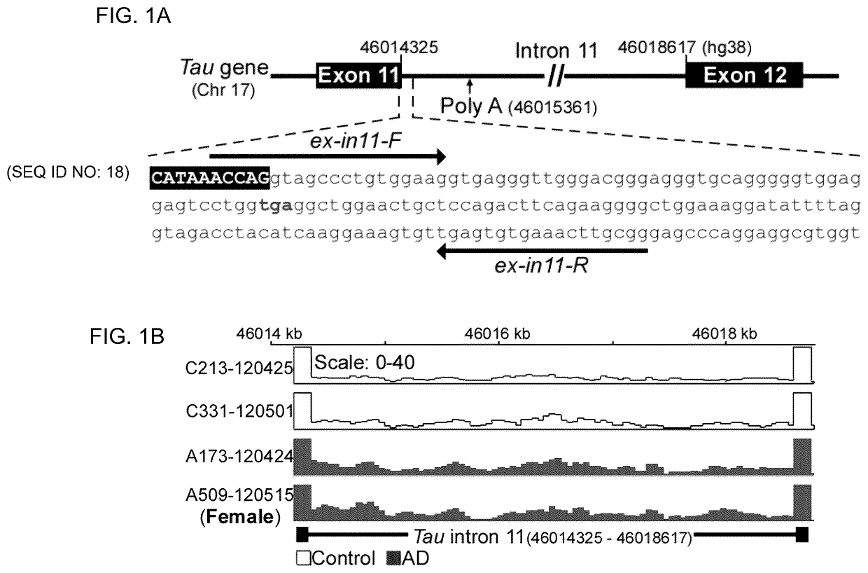

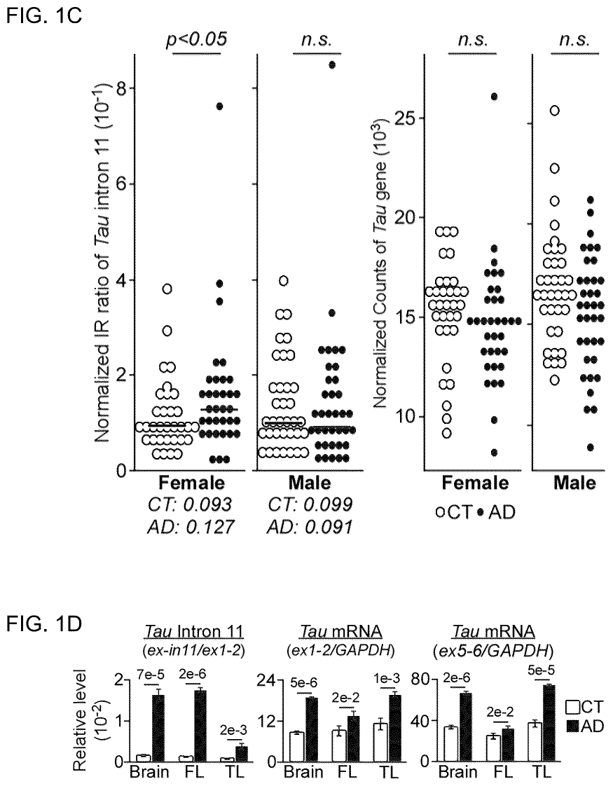

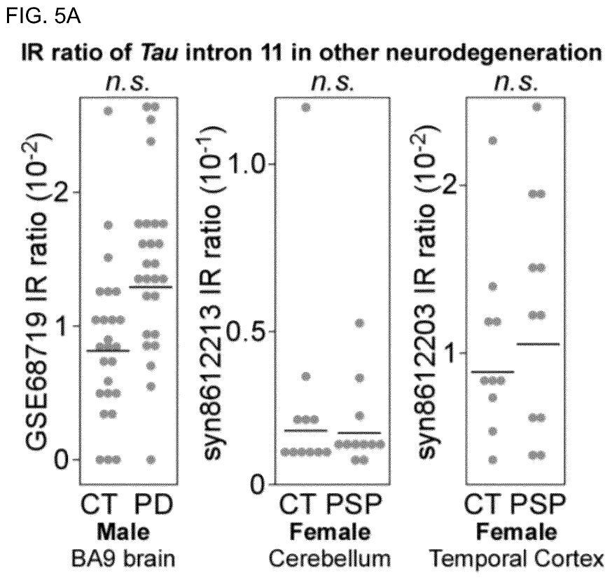

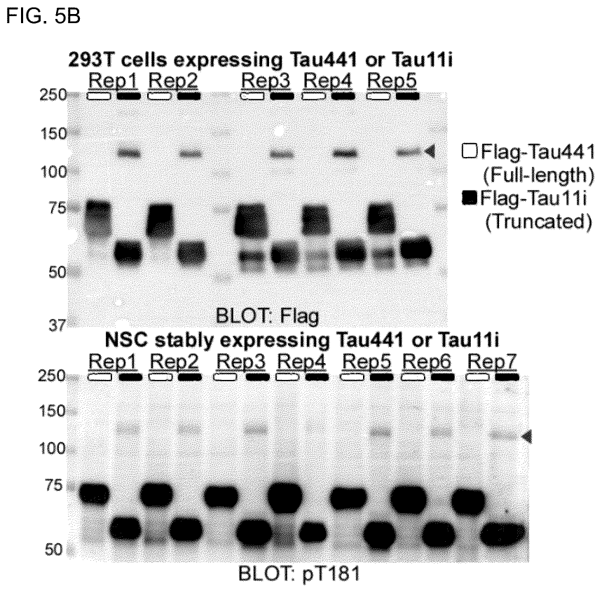

Analysis of more than 30 pairs of healthy controls and AD dorsolateral prefrontal cortex (DLPFC) (de Jager et al., Scientific data, 5 (1), 1-13 (2018)) using IRFinder software (Middleton et al., Genome Biol., 18, 1-11 (2017)) revealed increased retention of Tau intron 11 (FIG. 1A). These changes appeared to be unique to AD since no significant difference in IR ratio was observed in PD (Dumitriu et al., BMC Genom., 9 (1), 1-17 (2016)) and PSP (Allen et al., Sci. Data, 3 (1), 1-10 (2016)) datasets (FIG. 5A). Increased IR in AD samples was next validated by qPCR of brain, frontal, and temporal lobe from three distinct pairs of control and AD patients (FIG. 1D). The marked differences observed in qPCR could be explained by targeted amplification of intron 11 as compared to RNA-sequencing of the entire transcriptome where low abundance intronic read counts may be underrepresented. Although qPCR detected an elevated level of Tau mRNA in the three AD brain tissues samples as compared to control (FIG. 1D), no differential expression of Tau gene in the DLPFC (FIG. 1C) was observed.

### Example 3: Truncated Tau11i Oligomerizes and is More Enriched in Sarkosyl-Insoluble Fraction

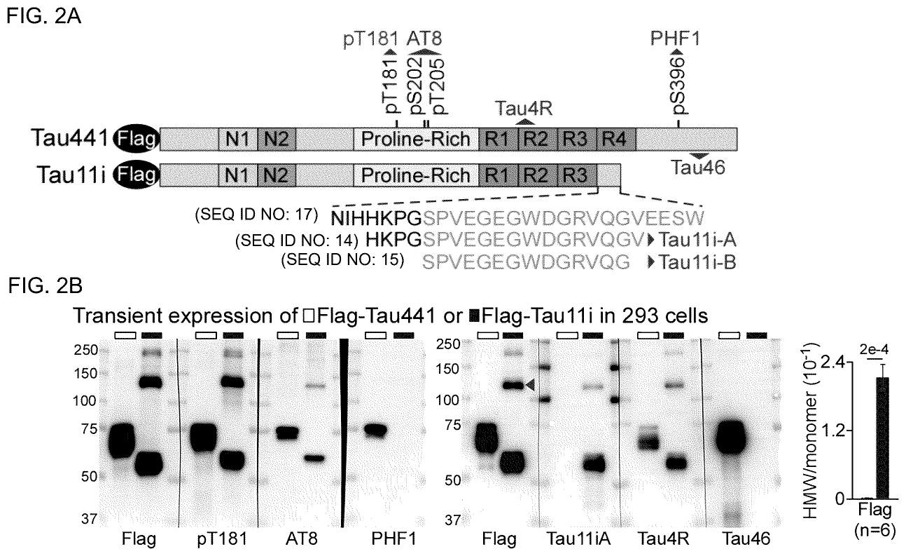

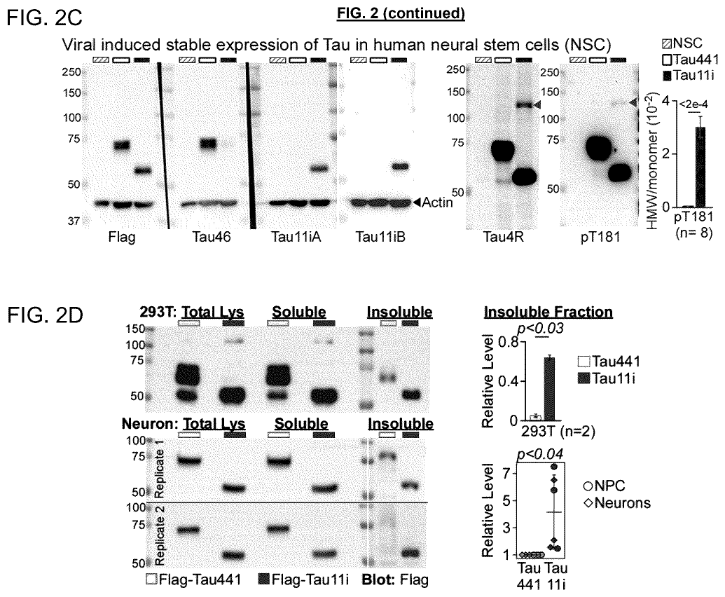

In the brain, full-length Tau encodes for 441 amino acids (herein Tau441) whereas the premature stop codon in intron 11 (FIG. 1A) may lead to protein truncation (herein Tau11i, amino acids encoded by retained intron (FIG. 2A, light grey text). The presence of canonical polyadenylation site 1 kb downstream of stop codon within intron 11 further suggests that the Tau mRNA transcript with IR might escape nonsense-mediated decay (NMD). To study the effect caused by IR, either Flag-tag full-length Tau441 or truncated Tau11i was expressed in 293T cells. As predicted, truncated Tau11i (FIG. 2B right well) runs below Tau441 (FIG. 2B left well) at ~55 kD size and cannot be detected by PHF1 or Tau46 antibodies which recognize Tau C-terminal domain. Importantly, it can be detected specifically by Tau11i-A antibody raised against the amino acid sequences encoded by the retained intron 11 (FIG. 2B). Interestingly, Tau11i protein also migrates as higher molecular weight (HMW) species on denaturing gels and was readily detected by pT181antibody, suggesting it undergoes oligomerization in 293T cells (FIGS. 2B and 5B). This observation is also consistent with the C-terminal domain inhibiting polymerization of Tau monomer (Martins et al., PloS one, 7 (4), e35461 (2012); Abraha et al., J. Cell Sci., 113 (21), 3737-3745 (2000); Gu et al., J Biol Chem, 295 (40), 13812-13828 (2020); Wang et al. Proc. Natl. Acad. Sci. USA, 104 (24), 10252-10257 (2007)) and HMW-Tau undergoes phosphorylation at Thr181 (Zhou et al., Front. Aging Neurosci., 10, 27 (2018)). To negate the effect of transient expression, lentiviral infection was used to stably express Flag-Tau441 or Flag-Tau11i in human neural stem cells (NSC). Similar to 293T cells, Flag-Tau11i protein was specifically recognized by two different Tau11i antibodies (Tau11i-A and Tau11i-B) but not Tau46 (FIG. 2C). Tau11i also consistently oligomerizes to form HMW species in NSC, albeit at a lower level, as detected by Tau4R and pT181 antibodies (FIGS. 2C and 5B). As formation of HMW species is one of the characteristics of pathological Tau in AD, other biochemical properties of Tau11i were further examined in these cells. Extraction by ionic detergent N-lauryl-sarcosine (Sarkosyl), which solubilizes natively folded protein effectively, has been widely used to isolate detergent-insoluble protein aggregates from brain tissues with neurodegenerative diseases such as AD (Diner et al., J. Vis. Exp., (128), e55835. (2017)). Compared to Flag-Tau441, there was more ectopically expressed Flag-Tau11i in the Sarkosyl-insoluble fraction from 293T cells (FIG. 2D top blot, right lane), neuronal progenitor cells (NPC, FIG. 2D middle blot) and neurons (FIG. 2D bottom blot). Taken together, the results indicate that ectopically expressed Tau11i undergoes multiple phosphorylation, oligomerizes to form HMW species and is enriched in Sarkosyl-insoluble fractions.

### Example 4: Detection of a Higher Level of Tau11i Species in AD Brain Regions

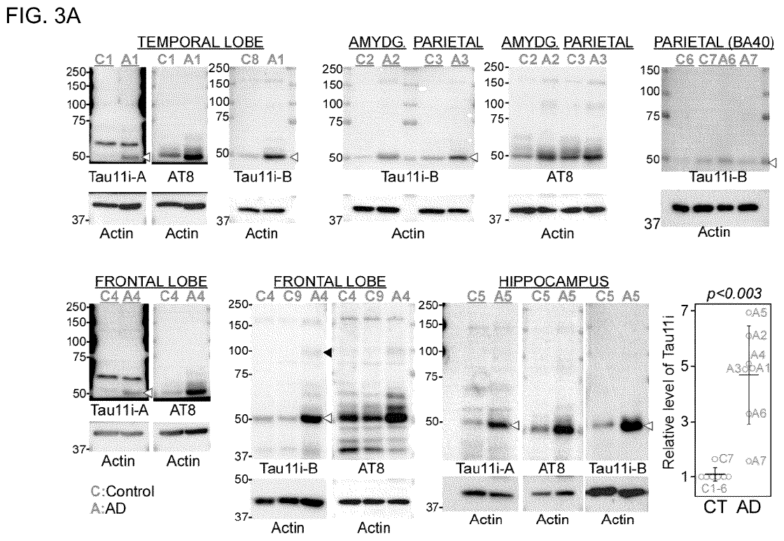

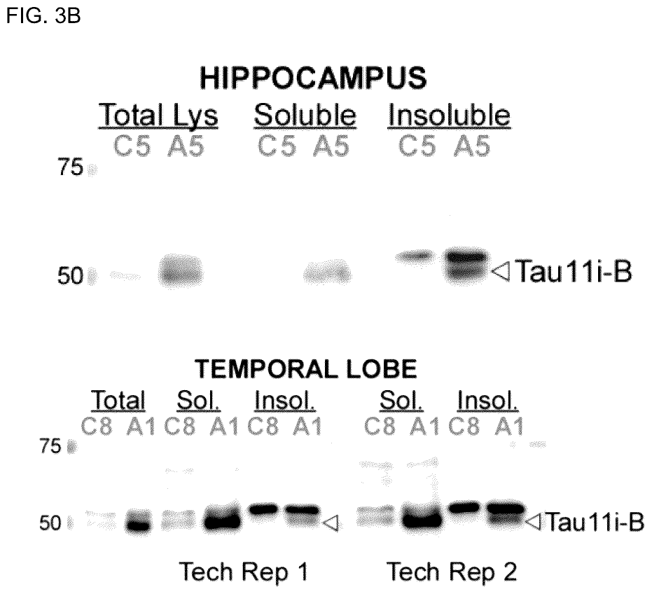

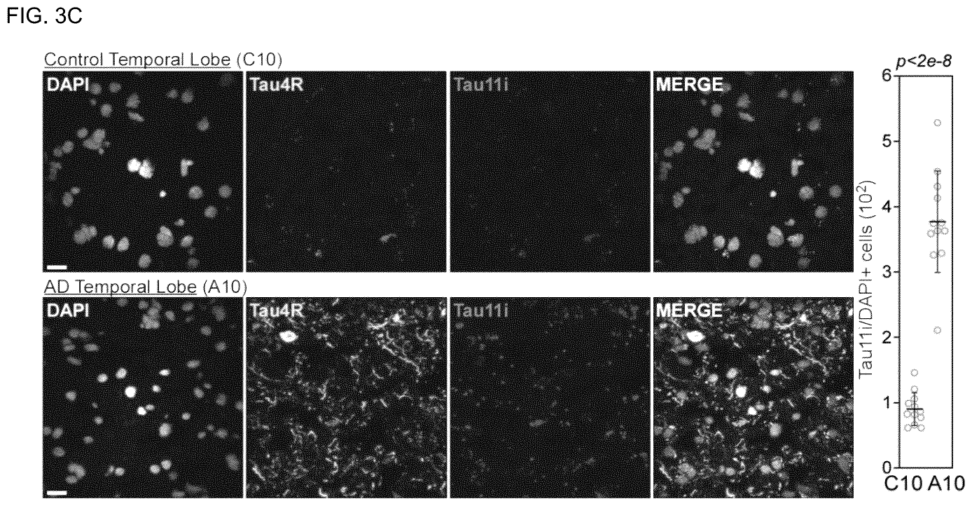

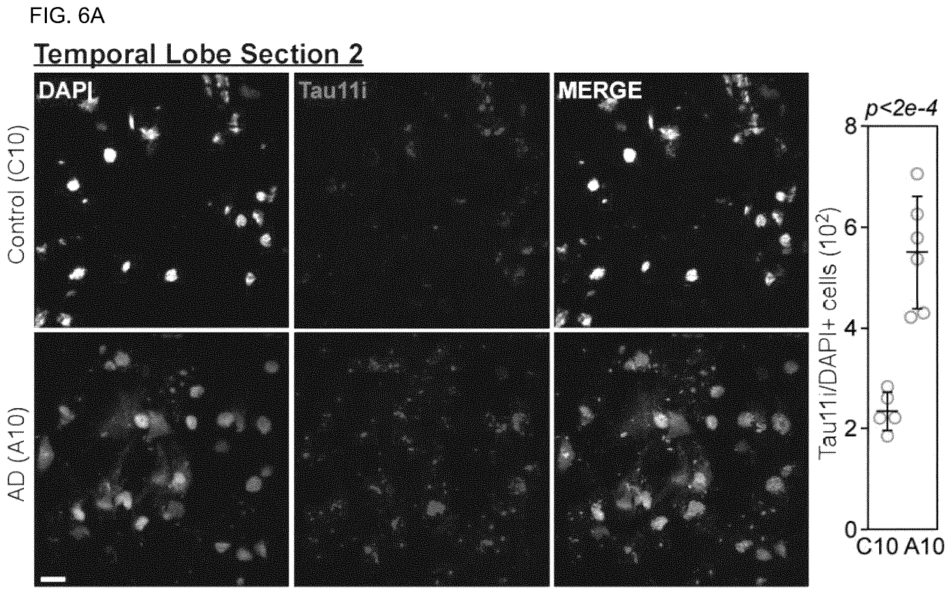

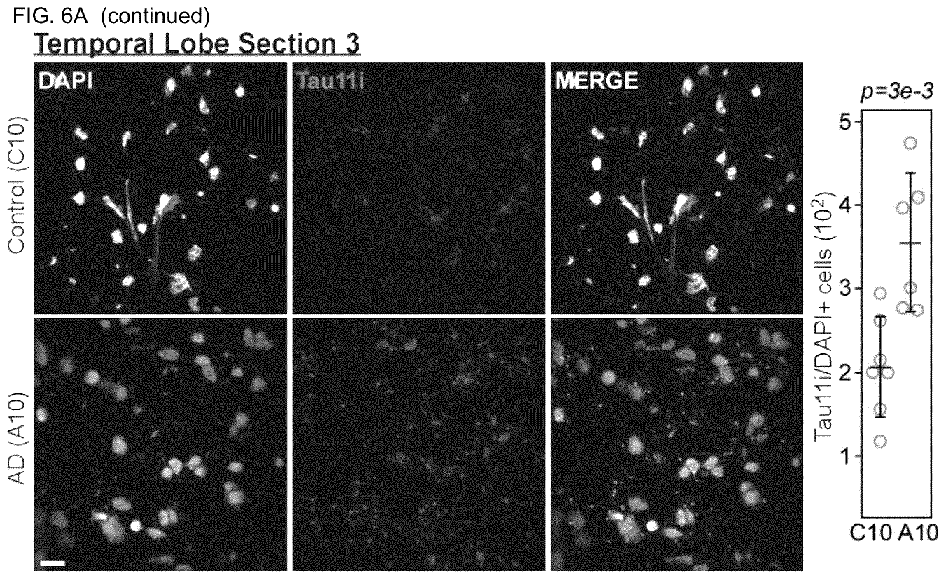

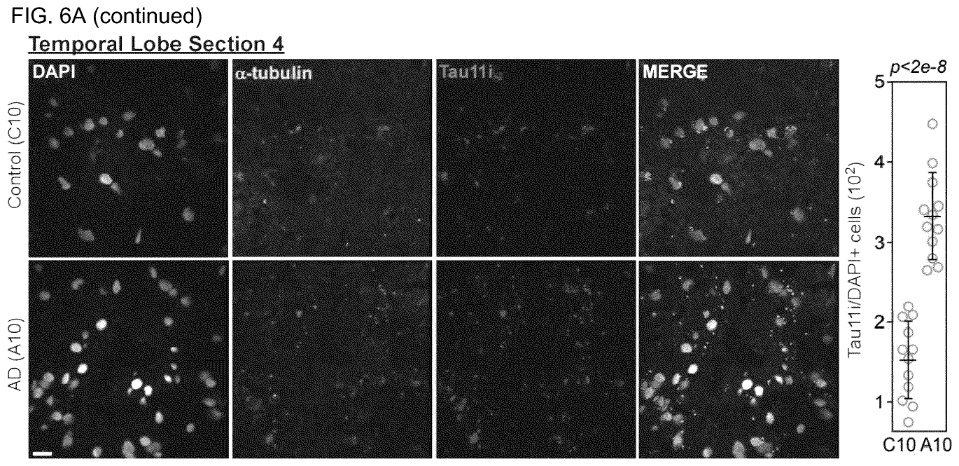

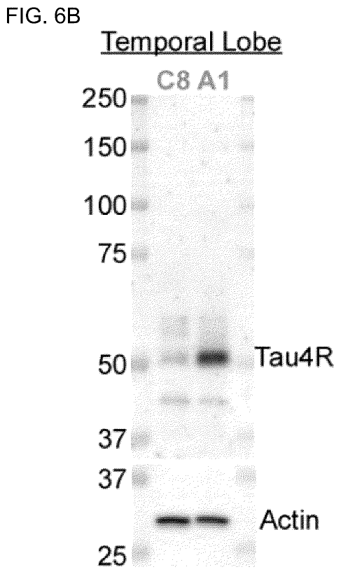

To test whether Tau mRNA transcript with IR is degraded by NMD or translated in vivo, hippocampus, amygdala, parietal, frontal, and temporal lobes from 9 healthy controls and 7 AD patients were probed with Tau11i and AT8 antibodies. Consistent with the increased retention of intron 11 in AD samples (FIGS. 1A-1D), two different Tau11i antibodies detected significantly higher level of ~50 kD band, which corresponds to AT8 band (phosphor-Tau at S202/T205), in the AD samples when compared to controls (FIG. 3A, white arrowhead. p<0.003, paired 2-tailed t-test, n=7). Although Tau11i antibodies failed to detect HMW form in NSC (FIG. 2C), weak higher migrating bands of different sizes were observed that correspond to AT8 immunoblot in AD frontal lobe lysates (FIG. 3A, black arrowhead). These results indicate that Tau mRNA with retained intron 11 escapes NMD and is translated into protein. The presence of premature stop codon and downstream canonical polyadenylation signal in intron 11 is likely to facilitate NMD evasion and the subsequent translation of novel truncated Tau11i protein in vivo. The higher level of Tau11i protein in AD tissues examined corroborates with an increased IR observed with transcriptomic analysis and qPCR validation. The similar migrating pattern on denaturing gel further suggests that Tau11i protein may undergo phosphorylation at serine 202 and threonine 205 (AT8) and oligomerize in AD tissues. Next, the solubility of endogenous Tau11i protein was investigated by Sarkosyl extraction. Compared to AD temporal lobe, a higher portion of Tau11i protein is found to be enriched in Sarkosyl-insoluble fraction in AD hippocampus (FIG. 3B, white arrow). This suggests that IR-induced C-terminal truncation may reduce the solubility of monomeric Tau11i protein in AD brain tissues. To better understand the cellular localization patterns of Tau11i in brain tissues, we stained control and AD temporal lobe sections with Tau11i and Tau4R (recognizes amino acid 275-291) (FIG. 3C) or α-tubulin (FIG. 6). Consistent with immunoblot result, there was higher level of Tau11i staining in AD as compared to control temporal lobe (FIG. 3C and FIGS. 6A-6B). Interestingly, Tau11i appears to form granular aggregates that co-localize weakly with Tau4R fibril-like structures in AD temporal lobe, suggesting that Tau11i might contribute to pre-tangle assembly rather than pathological neurofibrillary tangle burden (FIG. 3C).

### Example 5: Tau11i has Reduced Association with Microtubule and Increased Protein Stability

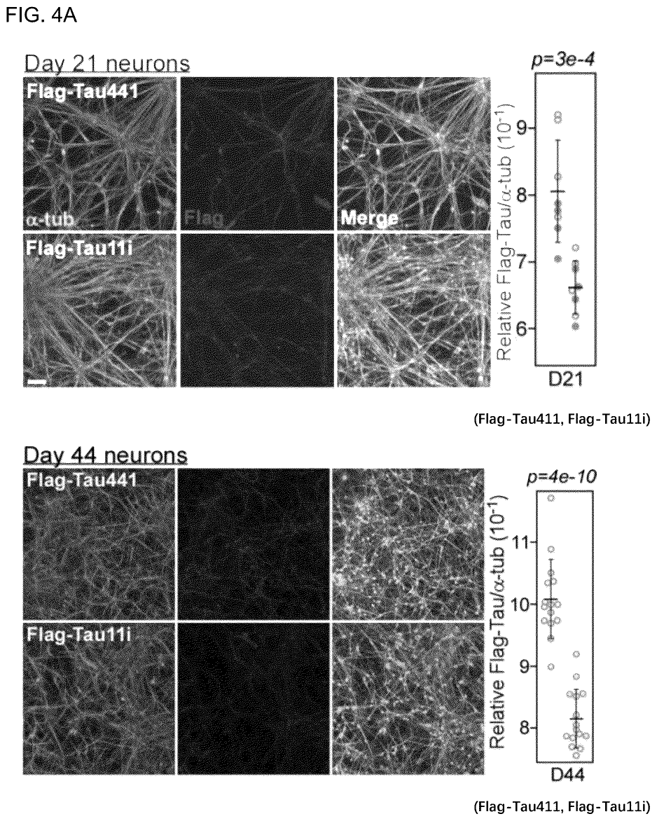

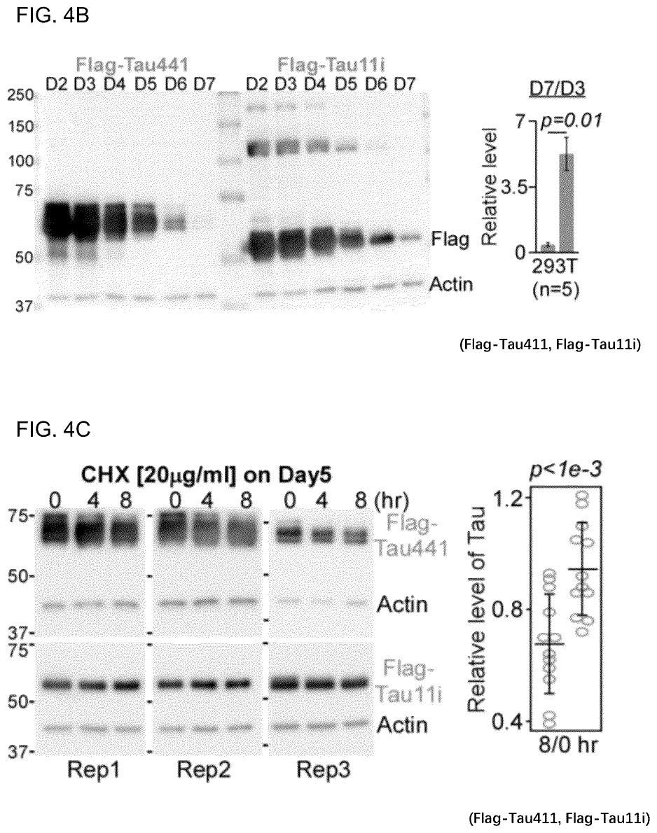

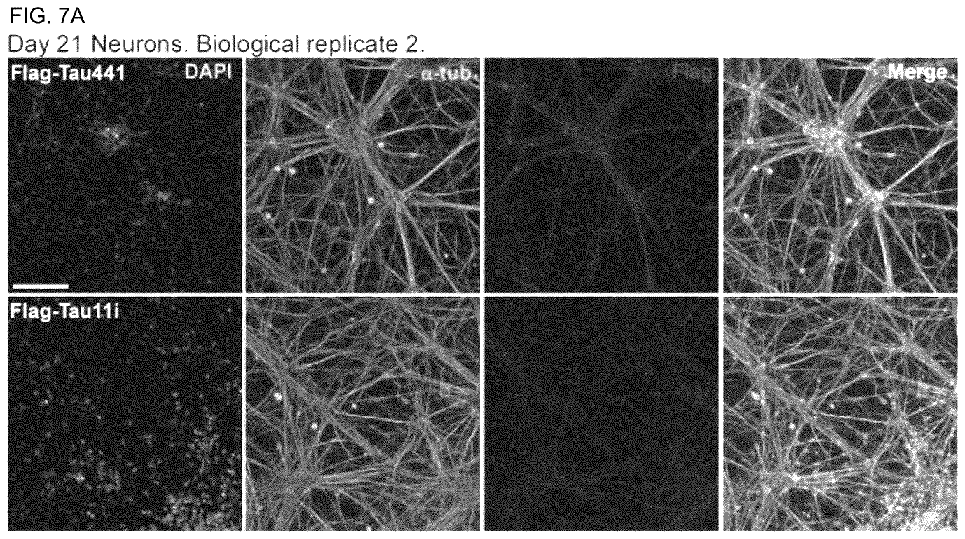

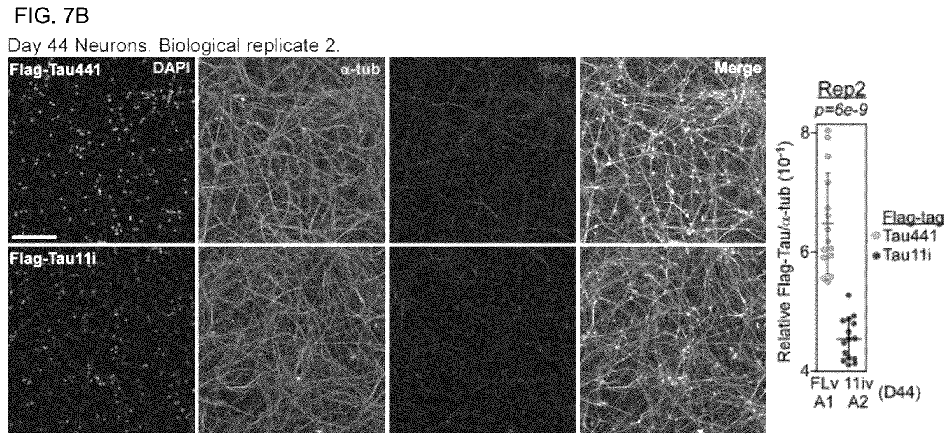

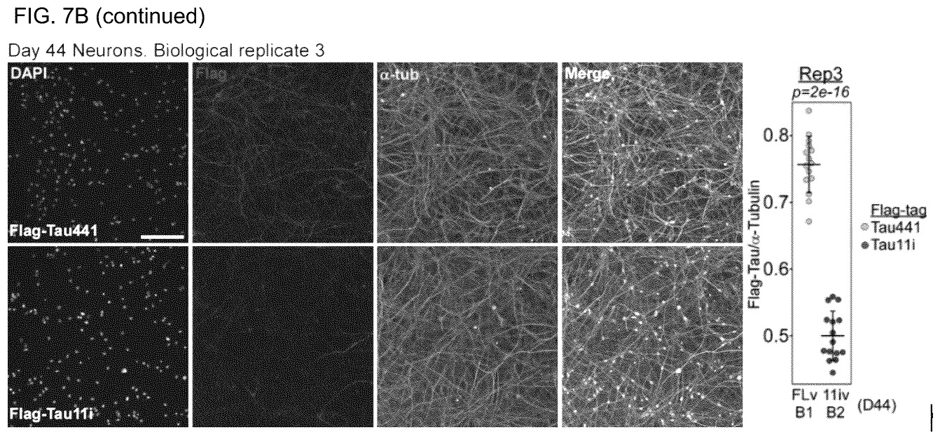

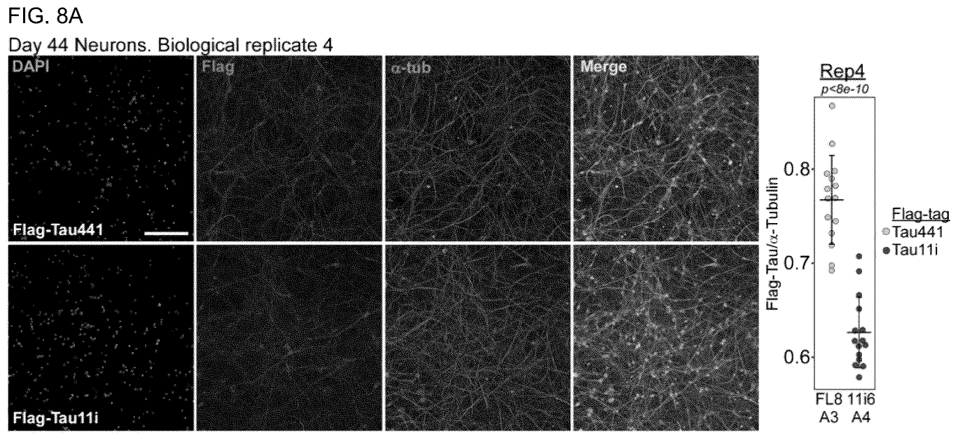

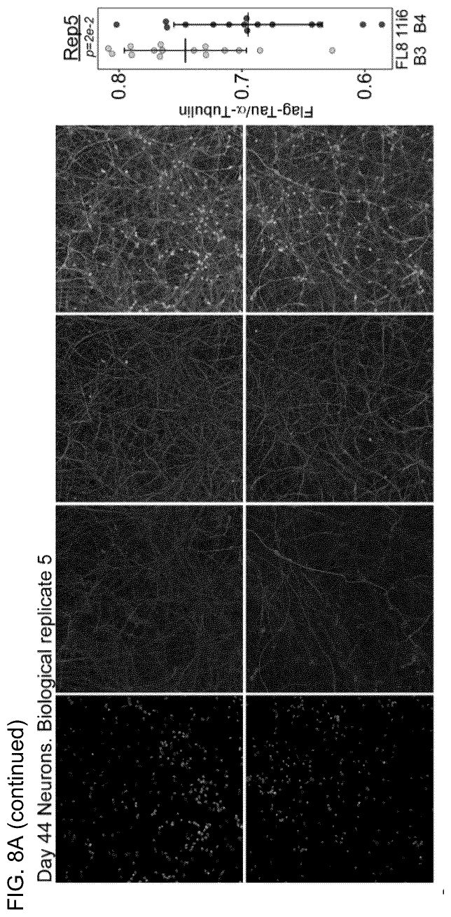

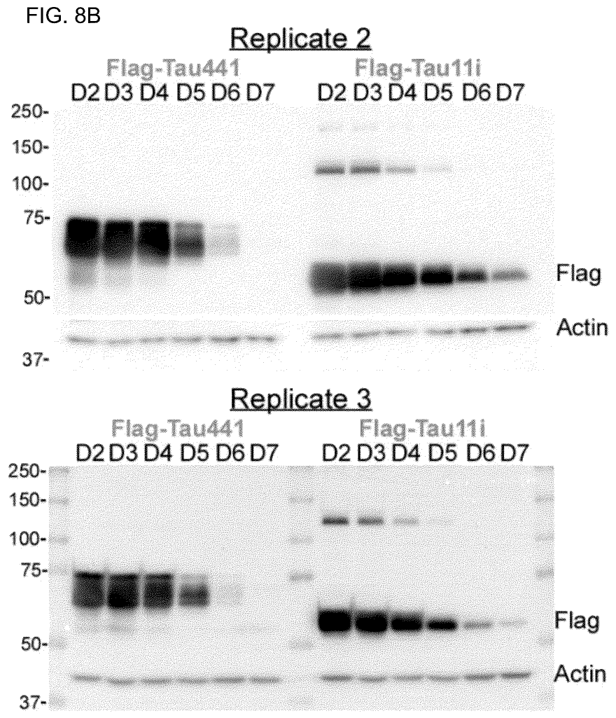

The association of Tau to microtubule can be disrupted by extensive phosphorylation and mutations. Therefore, it was investigated whether IR-induced protein truncation may impair its association with microtubules in neurons as well as alter its protein stability in cells. NSC expressing Flag-Tau441 and Tau11i were differentiated into cortical neurons (Day 14) and cultured for an additional one week (Day 21, n=2) or one month (Day 44, n=5). Immunostaining with anti-Flag and anti-α-tubulin antibodies showed a significant reduction in the colocalization signals of Tau11i with microtubule networks in neurons as compared to Tau441 (FIG. 4A, FIGS. 7A-7B and FIG. 8A), suggesting that Tau11i has lower binding efficiency to microtubules. The stability of Tau441 and Tau11i proteins was next investigated by time-course monitoring and cycloheximide assay of transiently transfected Tau protein. The level of Tau11i protein on Day 7 was around ~5% of that at Day 3 whereas Tau441 level dropped to about 0.4% (FIG. 4B and FIG. 8B). Consistent with time-course experiment, Tau11i was also more stable and exhibited less protein degradation than Tau441 following 8 h of cycloheximide treatment (FIG. 4C).

Characterization of Tau11i in different cell types revealed several biochemical properties that resemble pathological Tau (Gotz et al., Annu Rev Pathol, 14, 239-261 (2019); Iqbal et al., Nat. Rev. Neurol., 12 (1), 15-27. (2016)). Compared to full-length Tau441, truncated Tau11i oligomerizes to form HMW species and is enriched in Sarkosyl-insoluble fractions (Example 3), has reduced association with microtubule networks in neurons and exhibits lower rate of protein turnover (Example 5).

Analysis of different brain regions from distinct donors by qPCR, immunoblot and immunofluorescence showed that Tau11i protein was significantly elevated in AD tissues as compared to healthy control. In AD hippocampus, endogenous Tau11i is also observed in Sarkosyl-insoluble fraction, consistent with its association with protein aggregates. Examples in this disclosure indicate that Tau11i is a new biomarker for AD. Antibodies and nuclear acid detection methods demonstrated herein are therefore suitable for use in diagnostic purposes or assessing an individual at risk of developing AD.

### Example 6: Retention of Tau Intron 11 and Tau11i Protein are Detectable in Human Plasma

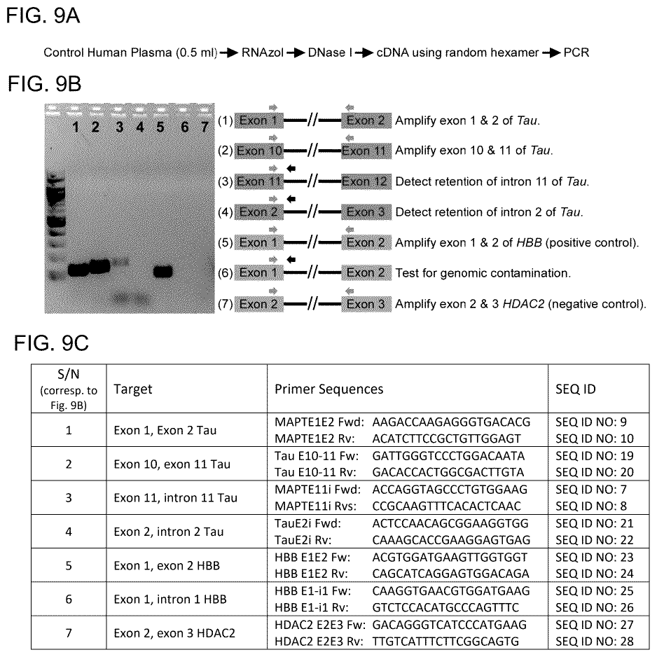

To test whether retention of Tau intron 11 is detectable in human plasma, 0.5 ml of control human plasma was lysed with 1.25 ml of RNAzol RT (Sigma). Total RNA was extracted and purified according to manufacturer's instructions. High-capacity cDNA reverse transcription kit (Applied Biosystems) was used to generate cDNA libraries from 1 μg of total RNA pre-treated with DNase I (Thermo Scientific). PCR was performed with Phusion DNA polymerase (NEB) with the primer pairs shown in FIG. 9C. Lane 3 of FIG. 9B shows a positive band representing an amplicon spanning the junction of Exon 11 and Intron 11, which indicates that retention of Tau intron 11 is detectable in human plasma.

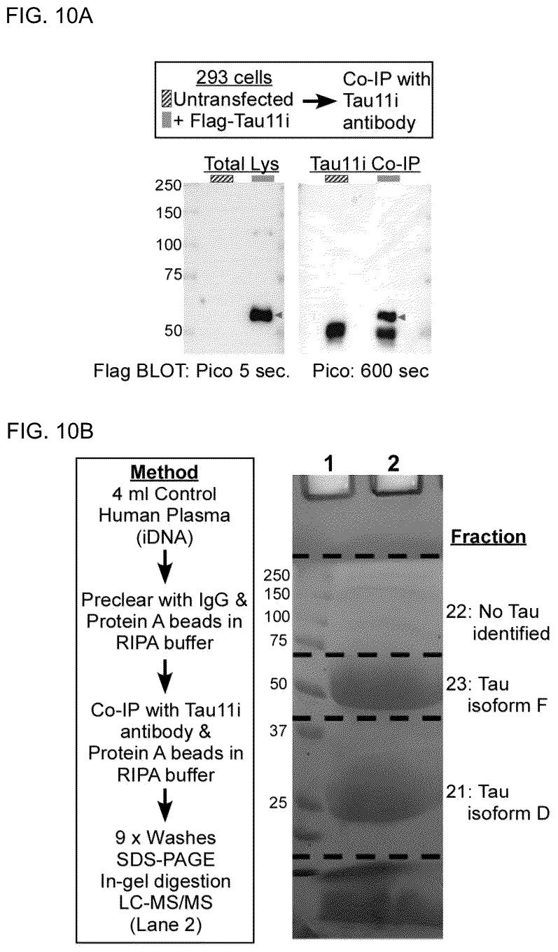

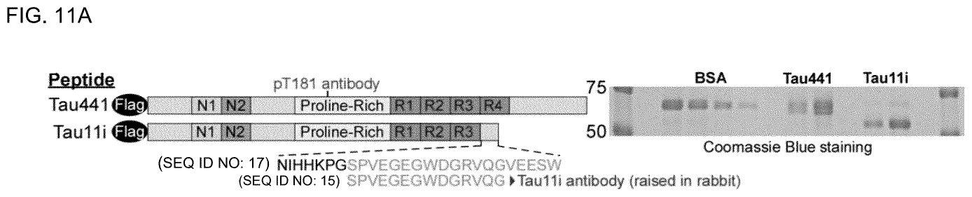

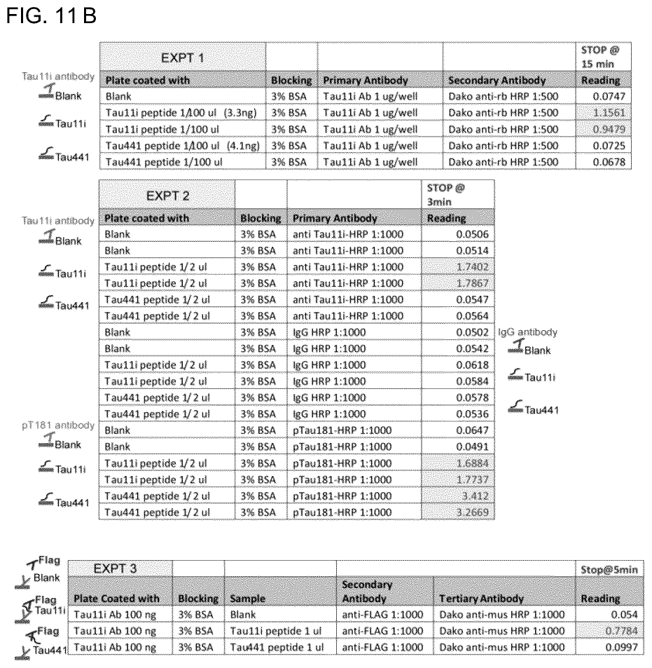

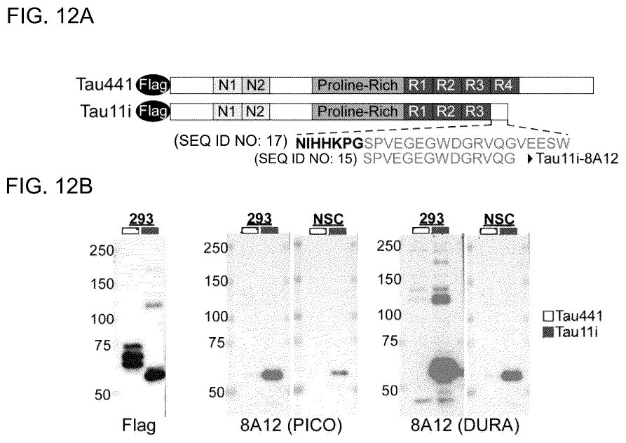

Similarly, by employing Tau11i-specific antibodies disclosed herein, Tau11i is also successfully detected in human plasma (FIGS. 10A-10B). Following Co-IP and SDS-PAGE electrophoresis, the fractions pulled down by the Tau11i-specific antibody, Tau11i-B, was then in-gel trypsin digested and subsequently subjected to LC-MS/MS.

Mass spectrometry data confirmed that the pull-down fractions correspond to Tau species. It is important to highlight that the reference database exclusively includes known variants of Tau. For example, Tau isoform F is Tau441 (441 amino acids) which contains 2N4R; isoform D has 383 amino acids which contains ON4R. Therefore, any newly identified forms, such as the one discussed in this disclosure, would not be encompassed in the database's records. Given that Tau11i-B is specific to Tau11i and does not bind to Tau441 (FIG. 2C), the above results indicate that detected in human plasma is Tau11i.

The presence of intron 11-retained Tau and Tau11i protein circulating in the bloodstream offers a unique opportunity for early and fast detection of such Tau abnormality. The accessibility of human plasma as a sample source greatly expedites the diagnostic process, eliminating the need for more invasive procedures that often pose risks and discomfort to patients. Furthermore, the identification and quantification of intron 11-retained Tau or Tau11i protein within human plasma can establish a direct link between their levels and the progression of AD pathologies, thereby enabling precise and prompt intervention.

### REFERENCES

Abraha, A., Ghoshal, N., Gamblin, T. C., Cryns, V., Berry, R. W., Kuret, J., & Binder, L. I. (2000). C-terminal inhibition of tau assembly in vitro and in Alzheimer's disease. Journal of cell science, 113 (21), 3737-3745.
Adusumalli, S., Ngian, Z. K., Lin, W. Q., Benoukraf, T., & Ong, C. T. (2019). Increased intron retention is a post-transcriptional signature associated with progressive aging and Alzheimer's disease. Aging cell, 18 (3), e12928.
Allen, M., Carrasquillo, M. M., Funk, C., Heavner, B. D., Zou, F., Younkin, C. S., Burgess, J. D., Chai, H. S., Crook, J., Eddy, J. A., Li, H., & Ertekin-Taner, N. (2016). Human whole genome genotype and transcriptome data for Alzheimer's and other neurodegenerative diseases. Scientific data, 3 (1), 1-10.
Ballabio, A., Ranier, J. E., Chamberlain, J. S., Zollo, M., & Caskey, C. T. (1990). Screening for steroid sulfatase (STS) gene deletions by multiplex DNA amplification. Human genetics, 84, 571-573.
Barany, F. (1991). Genetic disease detection and DNA amplification using cloned thermostable ligase. Proceedings of the National Academy of Sciences, 88 (1), 189-193.
Bustin, S. A. (2000). Absolute quantification of mRNA using real-time reverse transcription polymerase chain reaction assays. Journal of molecular endocrinology, 25 (2), 169-193.
Chamberlain, J. S., Gibbs, R. A., Rainer, J. E., Nguyen, P. N., & Thomas, C. (1988). Deletion screening of the Duchenne muscular dystrophy locus via multiplex DNA amplification. Nucleic acids research, 16 (23), 11141-11156.
Chua, X. Y., Chong, J. R., Cheng, A. L., Lee, J. H., Ballard, C., Aarsland, D., . . . & Lai, M. K. (2022). Elevation of inactive cleaved annexin A1 in the neocortex is associated with amyloid, inflammatory and apoptotic markers in neurodegenerative dementias. Neurochemistry International, 152, 105251.
De Jager, P. L., Ma, Y., McCabe, C., Xu, J., Vardarajan, B. N., Felsky, D., Klein, H. U., White., C. C., Peters, M. A., Lodgson B, Nejad, P., Bennett, D. A. (2018). A multi-omic atlas of the human frontal cortex for aging and Alzheimer's disease research. Scientific data, 5 (1), 1-13.
Diner, I., Nguyen, T., & Seyfried, N. T. (2017). Enrichment of detergent-insoluble protein aggregates from human postmortem brain. JoVE (Journal of Visualized Experiments), (128), e55835.
Don, R. H., Cox, P. T., Wainwright, B. J., Baker, K., & Mattick, J. S. (1991). ‘Touchdown’PCR to circumvent spurious priming during gene amplification. Nucleic acids research, 19 (14), 4008.
Dumitriu, A., Golji, J., Labadorf, A. T., Gao, B., Beach, T. G., Myers, R. H., Longo, K. A., & Latourelle, J. C. (2015). Integrative analyses of proteomics and RNA transcriptomics implicate mitochondrial processes, protein folding pathways and GWAS loci in Parkinson disease. BMC medical genomics, 9 (1), 1-17.
García-Escudero, V., Ruiz-Gabarre, D., Gargini, R., Pérez, M., García, E., Cuadros, R., Hernández I. H., Cabrera J. R., García-Escudero R., Lucas J. J., Hernández F., & Ávila, J. (2021). A new non-aggregative splicing isoform of human Tau is decreased in Alzheimer's disease. Acta Neuropathologica, 142, 159-177.
Gootenberg, J. S., Abudayyeh, O. O., Lee, J. W., Essletzbichler, P., Dy, A. J., Joung, J., Verdine, V., Donghia, N., Daringer, N. M., Freije, C. A., Myhrvold, C., Bhattacharyya, R. P., Livny, J., Regev, A., Koonin, E. V., Hung, D. T., Sabeti., P. C., Collins, J. J., & Zhang, F. (2017). Nucleic acid detection with CRISPR-Cas13a/C2c2. Science, 356 (6336), 438-442.
Götz, J., Halliday, G., & Nisbet, R. M. (2019). Molecular pathogenesis of the tauopathies. Annual Review of Pathology: Mechanisms of Disease, 14, 239-261.
Gregory, D., Hooker, J., & Alessandro, C. (2018). A dataset mapping the potential biophysical effects of vegetation cover change. Scientific Data, 5 (1).
Gu, J., Xu, W., Jin, N., Li, L., Zhou, Y., Chu, D., Gong, C. X., Iqbal, K., & Liu, F. (2020). Truncation of Tau selectively facilitates its pathological activities. Journal of Biological Chemistry, 295 (40), 13812-13828.
Guilfoyle, R. A., Leeck, C. L., Kroening, K. D., Smith, L. M., & Guo, Z. (1997). Ligation-mediated PCR amplification of specific fragments from a class-II restriction endonuclease total digest. Nucleic acids research, 25 (9), 1854-1858.
Hall, J. G., Eis, P. S., Law, S. M., Reynaldo, L. P., Prudent, J. R., Marshall, D. J., Allawi H. T., Mast A. L., Dahlberg J. E., Kwiatkowski R. W., de Arruda M., Neri B. P., Lyamichev, V. I. (2000). Sensitive detection of DNA polymorphisms by the serial invasive signal amplification reaction. Proceedings of the National Academy of Sciences, 97 (15), 8272-8277.
Hayden, M. J., Nguyen, T. M., Waterman, A., & Chalmers, K. J. (2008). Multiplex-ready PCR: a new method for multiplexed SSR and SNP genotyping. BMC genomics, 9, 1-12.
Hecker, K. H., & Roux, K. H. (1996). High and low annealing temperatures increase both specificity and yield in touchdown and stepdown PCR. Biotechniques, 20 (3), 478-485.
Herman, J. G., Graff, J. R., Myöhanen, S. B. D. N., Nelkin, B. D., & Baylin, S. B. (1996). Methylation-specific PCR: a novel PCR assay for methylation status of CpG islands. Proceedings of the national academy of sciences, 93 (18), 9821-9826.
Higuchi, R., Krummel, B., & Saiki, R. (1988). A general method of in vitro preparation and specific mutagenesis of DNA fragments: study of protein and DNA interactions. Nucleic acids research, 16 (15), 7351-7367.
Higuchi, R., Dollinger, G., Walsh, P. S., & Griffith, R. (1992). Simultaneous amplification and detection of specific DNA sequences. Bio/technology, 10 (4), 413-417.
Higuchi, R., Fockler, C., Dollinger, G., & Watson, R. (1993). Kinetic PCR analysis: real-time monitoring of DNA amplification reactions. Bio/technology, 11 (9), 1026-1030.
Iqbal, K., Liu, F., & Gong, C. X. (2016). Tau and neurodegenerative disease: the story so far. Nature reviews neurology, 12 (1), 15-27.
Kalinina, O., Lebedeva, I., Brown, J., & Silver, J. (1997). Nanoliter scale PCR with TaqMan detection. Nucleic acids research, 25 (10), 1999-2004.
Love, M. I., Huber, W., & Anders, S. (2014). Moderated estimation of fold change and dispersion for RNA-seq data with DESeq2. Genome biology, 15 (12), 1-21.
Lyamichev, V., Mast, A. L., Hall, J. G., Prudent, J. R., Kaiser, M. W., Takova, T., & Brow, M. A. D. (1999). Polymorphism identification and quantitative detection of genomic DNA by invasive cleavage of oligonucleotide probes. Nature biotechnology, 17 (3), 292-296.
Martins, R., Proenca, D., Silva, B., Barbosa, C., Silva, A. L., Faustino, P., & Romao, L. (2012). Alternative polyadenylation and nonsense-mediated decay coordinately regulate the human HFE mRNA levels. PloS one, 7 (4), e35461.
Middleton, R., Gao, D., Thomas, A., Singh, B., Au, A., Wong, J. J., Bomane, A., Cosson, B., Eyras, E., Rasko, J. E., & Ritchie, W. (2017). IRFinder: assessing the impact of intron retention on mammalian gene expression. Genome biology, 18, 1-11.
Rao, V. K., Swarnaseetha, A., Tham, G. H., Lin, W. Q., Han, B. B., Benoukraf, T., Xu, G. L., & Ong, C. T. (2020). Phosphorylation of Tet3 by cdk5 is critical for robust activation of BRN2 during neuronal differentiation. Nucleic Acids Research, 48 (3), 1225-1238.
Roux K H. (1994). Using mismatched primer-template pairs in touchdown PCR. Bio Techniques 16:812-814
Schouten, J. P., McElgunn, C. J., Waaijer, R., Zwijnenburg, D., Diepvens, F., & Pals, G. (2002). Relative quantification of 40 nucleic acid sequences by multiplex ligation-dependent probe amplification. Nucleic acids research, 30 (12), e57-e57.
Smith, A. R., Wheildon, G., & Lunnon, K. (2020). Invited Review—A 5-year update on epigenome-wide association studies of DNA modifications in Alzheimer's disease: progress, practicalities and promise. Neuropathology and Applied Neurobiology, 46 (7), 641-653.
Teng, I. T., Li, X., Yadikar, H. A., Yang, Z., Li, L., Lyu, Y., . . . & Tan, W. (2018). Identification and characterization of DNA aptamers specific for phosphorylation epitopes of tau protein. Journal of the American Chemical Society, 140 (43), 14314-14323.
Triglia, T., Peterson, M. G., & Kemp, D. J. (1988). A procedure for in vitro amplification of DNA segments that lie outside the boundaries of known sequences. Nucleic Acids Research, 16 (16), 8186.
Trabzuni, D., Wray, S., Vandrovcova, J., Ramasamy, A., Walker, R., Smith, C., Luk, C., Gibbs, J. R., Dillman, A., Hernandez, D. G., Arepalli, S., Singleton, A. B., Cookson, M. R., Pittman, A. M., de Silva, R, Weale, M. E., Hardy, J., & Ryten, M. (2012). MAPT expression and splicing is differentially regulated by brain region: relation to genotype and implication for tauopathies. Human molecular genetics, 21 (18), 4094-4103.
Vogelstein, B., & Kinzler, K. W. (1999). Digital pcr. Proceedings of the National Academy of Sciences, 96 (16), 9236-9241.
Wang, Y. P., Biernat, J., Pickhardt, M., Mandelkow, E., & Mandelkow, E. M. (2007). Stepwise proteolysis liberates tau fragments that nucleate the Alzheimer-like aggregation of full-length tau in a neuronal cell model. Proceedings of the National Academy of Sciences, 104 (24), 10252-10257.
Zhou, Y., Shi, J., Chu, D., Hu, W., Guan, Z., Gong, C. X., . . . & Liu, F. (2018). Relevance of phosphorylation and truncation of tau to the etiopathogenesis of Alzheimer's disease. Frontiers in aging neuroscience, 10, 27.

1. An isolated or recombinant Tau11i-derived polypeptide comprising the following amino acid sequence:
1) the amino acid sequence HKPGSPVEGEGWDGRVQGV, as set forth in SEQ ID NO: 14 or antigenic fragment thereof, or
2) the amino acid sequence SPVEGEGWDGRVQG, as set forth in SEQ ID NO: 15, or
3) an amino acid sequence resulting from substitution, deletion, addition or insertion of one or two or more amino acids in the amino acid sequence set forth in SEQ ID NO: 14 or SEQ ID NO: 15.

2. The isolated or recombinant Tau11i-derived polypeptide according to claim 1, wherein the polypeptide consists of the amino acid sequence set forth in SEQ ID NO: 1 or SEQ ID NO: 2.

3. An isolated antibody or antigen-binding fragment thereof that specifically binds to the polypeptide comprising the amino acid sequence HKPGSPVEGEGWDGRVQGV (SEQ ID NO: 14) or antigenic fragment thereof, or the amino acid sequence SPVEGEGWDGRVQG, (SEQ ID NO: 15), or an amino acid sequence resulting from substitution, deletion, addition or insertion of one or two or more amino acids in the amino acid sequence set forth in SEQ ID NO: 14 or SEQ ID NO: 15.

4. The isolated antibody or antigen-binding fragment thereof according to claim 3, wherein the polypeptide consists of the amino acid sequence set forth in SEQ ID NO: 1 or SEQ ID NO: 2.

5. The isolated antibody or antigen-binding fragment thereof according to claim 3, wherein the antibody or antigen-binding fragment thereof comprises a heavy chain variable region comprising complementarity determining regions HCDR1 having an amino acid sequence set forth in SEQ ID NO: 31, HCDR2 having an amino acid sequence set forth in SEQ ID NO: 32, and HCDR3 having an amino acid sequence set forth in SEQ ID NO: 33, and/or a light chain variable region comprising complementarity determining regions LCDR1 having an amino acid sequence set forth in SEQ ID NO: 36, LCDR2 having an amino acid sequence set forth in SEQ ID NO:37, and LCDR3 having an amino acid sequence set forth in SEQ ID NO: 38.

6. The isolated antibody or antigen-binding fragment thereof according to claim 5, wherein the antibody or antigen-binding fragment thereof comprises a heavy chain variable region having the amino acid sequence set forth in SEQ ID NO: 29 and/or a light chain variable region having the amino acid sequence set forth in SEQ ID NO: 34.

7. An oligonucleotide for amplifying a polynucleotide sequence spanning the junction of exon 11 and intron 11 of a Tau gene.

8. The oligonucleotide according to claim 7, wherein the oligonucleotide consists of the nucleotide sequence set forth in SEQ ID NO: 7 or SEQ ID NO: 8.

9. A polynucleic acid molecule encoding:
i) the polypeptide according to claim 1, or
ii) an isolated antibody or antigen-binding fragment thereof that specifically binds to the polypeptide comprising the amino acid sequence HKPGSPVEGEGWDGRVQGV (SEQ ID NO: 14) or antigenic fragment thereof, or the amino acid sequence SPVEGEGWDGRVQG, (SEQ ID NO: 15), or an amino acid sequence resulting from substitution, deletion, addition or insertion of one or two or more amino acids in the amino acid sequence set forth in SEQ ID NO: 14 or SEQ ID NO: 15 the antibody or antigen binding fragment thereof according to any one of claims 3 to 6.

10. (canceled)

11. A method of detecting the polypeptide according to claim 1, the method comprises contacting a sample suspected of comprising the polypeptide with an isolated antibody or antigen-binding fragment thereof that specifically binds to the polypeptide comprising the amino acid sequence HKPGSPVEGEGWDGRVQGV (SEQ ID NO: 14) or antigenic fragment thereof, or the amino acid sequence SPVEGEGWDGRVQG, (SEQ ID NO: 15), or an amino acid sequence resulting from substitution, deletion, addition or insertion of one or two or more amino acids in the amino acid sequence set forth in SEQ ID NO: 14 or SEQ ID NO: 15.

12. A method for diagnosing Alzheimer's disease or identifying a subject at risk of developing Alzheimer's disease, wherein the method comprises:
(a) contacting a sample derived from the subject with the antibody or antigen binding fragment thereof according to claim 3; or
(b) conducting a nucleic acid detection assay to assess the abundance of intron 11 retaining mRNA in a sample derived from the subject; or
(c) contacting a sample derived from the subject with the Tau11i epitope-specific aptamer that specifically binds to a polypeptide comprising the amino acid sequence set forth in any one of SEQ ID NOs: 1, 2, 13, 14 or 15.

13. The method according to claim 12, wherein the nucleic acid detection assay in (b) comprises amplifying a polynucleotide sequence spanning the junction of exon 11 and intron 11 of a Tau gene.

14. The method according to claim 13, wherein the amplification comprises using oligonucleotides having the nucleotide sequences as set forth in SEQ ID NOs: 7 and/or SEQ ID NO: 8.

15. The method according to claim 11, wherein the sample is brain tissue, cerebrospinal fluid, blood plasma, saliva, lacrimal fluid or urine.

16. A method of screening a candidate therapeutic or prophylactic drug for Alzheimer's disease, comprising using any of the following as an index:
a) reduction of the amount of Tau protein in the body of an Alzheimer's disease subject or a subject at risk of developing Alzheimer's disease, or
b) inhibition of production of Tau protein in the body of an Alzheimer's disease subject or a subject at risk of developing Alzheimer's disease,
wherein, the Tau protein comprises the amino acid sequence of the polypeptide according to claim 1.

17. A method of preparing a Tau11i-specific antibody, comprising
a) injecting the polypeptide according to claim 1 into an animal to induce an immune response to the polypeptide;
b) isolating antibodies that bind to the polypeptide;
c) selecting antibodies of (b) that binds to Tau11i but do not bind to full length Tau thereby producing an Tau11i-specific antibody.

18. The method according to claim 17, comprising
a) injecting at least one non-human mammal with at least one Tau11i-derived polypeptide to form at least one B cell specific for Tau11i, wherein the at least one Tau11i-derived polypeptide comprising the following amino acid sequence:
1) the amino acid sequence HKPGSPVEGEGWDGRVQGV, as set forth in SEQ ID NO: 14 or antigenic fragment thereof, or
2) the amino acid sequence SPVEGEGWDGRVQG, as set forth in SEQ ID NO: 15, or
3) an amino acid sequence resulting from substitution, deletion, addition or insertion of one or two or more amino acids in the amino acid sequence set forth in SEQ ID NO: 14 or SEQ ID NO: 15;
b) selecting at least one B cell specific to the Tau11i;
c) fusing the selected B cell with at least one immortalized cell to produce at least one hybridoma, wherein the hybridoma is capable of producing at least one antibody or a fragment thereof specific for the Tau11i; and
d) optionally isolating the antibody from the hybridoma and sequencing the variable heavy and light chains.

19. A method of screening and/or selecting a Tau11i epitope-specific aptamer, the method comprises: conducting systematic evolution of ligands by exponential enrichment (SELEX) technique to identify Tau11i epitope-specific aptamers, wherein the SELEX technique comprises providing a polypeptide comprising the amino acid sequence set forth in any one of SEQ ID NOs: 1, 2, 13, 14 or 15 as a target ligand.

20. A Tau11i-specific aptamer capable of binding to a polypeptide comprising the amino acid sequence set forth in any one of SEQ ID NOs: 1, 2, 13, 14 or 15.

21. A kit comprising the antibody or antigen binding fragment thereof according to claim 3, and/or the isolated polypeptide.

22. (canceled)


SEQUENCE LISTING (38 sequences)

SEQ ID NO: 1 - protein, 20 aa, synthetic construct
HKPGSPVEGEGWDGRVQGVC

SEQ ID NO: 2 - protein, 15 aa, synthetic construct
CSPVEGEGWDGRVQG

SEQ ID NO: 3 - DNA, 23 bp, synthetic construct
  source 1..23: mol_type = other DNA
ctatctagagctgagccccgcca

SEQ ID NO: 4 - DNA, 25 bp, synthetic construct
  source 1..25: mol_type = other DNA
aacgtcgactcacaaaccctgcttg

SEQ ID NO: 5 - DNA, 19 bp, synthetic construct
  source 1..19: mol_type = other DNA
gtgcaaatagtctacaaac

SEQ ID NO: 6 - DNA, 22 bp, synthetic construct
  source 1..22: mol_type = other DNA
gtcgactcaccaggactcctcc

SEQ ID NO: 7 - DNA, 20 bp, synthetic construct
  source 1..20: mol_type = other DNA
accaggtagccctgtggaag

SEQ ID NO: 8 - DNA, 20 bp, synthetic construct
  source 1..20: mol_type = other DNA
ccgcaagtttcacactcaac

SEQ ID NO: 9 - DNA, 20 bp, synthetic construct
  source 1..20: mol_type = other DNA
aagaccaagagggtgacacg

SEQ ID NO: 10 - DNA, 20 bp, synthetic construct
  source 1..20: mol_type = other DNA
acatcttccgctgttggagt

SEQ ID NO: 11 - DNA, 20 bp, synthetic construct
  source 1..20: mol_type = other DNA
caatgaccccttcattgacc

SEQ ID NO: 12 - DNA, 20 bp, synthetic construct
  source 1..20: mol_type = other DNA
gacaagcttcccgttctcag

SEQ ID NO: 13 - protein, 19 aa, Homo sapiens
SPVEGEGWDGRVQGVEESW

SEQ ID NO: 14 - protein, 19 aa, synthetic construct
HKPGSPVEGEGWDGRVQGV

SEQ ID NO: 15 - protein, 14 aa, synthetic construct
SPVEGEGWDGRVQG

SEQ ID NO: 16 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 17 - protein, 352 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGSPVEGEGWDGRVQGVEESW

SEQ ID NO: 18 - DNA, 4488 bp, Homo sapiens
  source 1..4488: mol_type = genomic DNA
gtgcaaatagtctacaaaccagttgacctgagcaaggtgacctccaagtgtggctcattaggcaacatccatcataaaccaggtagccctgtggaaggtgagggttgggacgggagggtgcagggggtggaggagtcctggtgaggctggaactgctccagacttcagaaggggctggaaaggatattttaggtagacctacatcaaggaaagtgttgagtgtgaaacttgcgggagcccaggaggcgtggtggctccagctcgctcctgcccaggccatgctgcccaagacaaggtgaggcgggagtgaagtgaaataaggcaggcacagaaagaaagcacatattctcggccgggcgctgtggctcacgcctgtaattccagcactttgggaggccaaggtgggtggatcatgaggtcaggagattgagaccatcctggctaacacagtgaaaccccgtctctactaaaaatacaaaaaattagccgggcgtggtggtgggcgcctgtagtcccagctactccggaggctgaggcaggaaaatggcgtgaacccggaaggcggagcttgcagtgagcggagtgagcagagatcgcgccactgcactccagcctgggcgacagagcgagactccgtctcaaaaaaaaaaagcacatgttctcgcttctttgtgggatccaggagatagagaatagaaggatggttaccagaggctgggaagggtagtgaggggatggtggggggatggtcaatgggtacaaaaaaaatagaataagacctagtatttgatagtgcaacagggtgactatagtcaataataatttaattgtacatttaaaaataactaaaagatagccgggtgcagtggcttacgtctgtaatcccagtactttgggaggctgaggtgggcgtttgagaccagcctggccaacatggtgaaaccccatctctactaaaaatacaaaaattagccaggcatggtggcgggcgcctgtaatcccagctactcgggaggctgaggcaggagaatcacttgaacctgggaggcagaggttgcagtgagccgagatcttgccactgcactccagcctgggtgacagtgaaactccgtctcaaaaataaaaataaaaatacagctgggcacggtggctcacgcctgtaatcccagcactttgggaggccgaggcgagcggatcacaaggtcaggagatatagaccatcctggctaacacggtgaaacccggtctctactaaaaatacaaaaaattagccaggcgtggtggcaggtgcctatagtcccagctactcacaaggctgaggcaggagaatggcatgaacctgggaggcggagcttgcagtgagccgagattgtgccactgcactccagcctgggcgagagagtgagactccgtctcaaaacaaaaacaaaaacaaaaacaaaaacaaacacacaacaaaaacctaaaagaatataaatggattgtttgtaacacaaaggacaaatgtttgaggggatggataccccattttccatgatgtgattattatacattgtgtgtctgtatcaaaacatctcatgagccccataaatatatacacctaactatgtacccacaaaaattaaaaaaatatattttttaaggtgaagagggaggcgagatgctggccttaacccctaacccgttgttctccctgcaagctgtccacagggcctctcagactcgaggttcagctatatggatgcatgagcttggtccccagccaacatgggagacacttcaccatcggcagcagctacagcacaggaaccctgggtcactgccatgtcccctctgtgactttgtttaaacagaaaatgatgctctgggccggctgtggtggcccacacctataatcccagcaccttgggaggcgggggtgggcagattgcctgaggtcaggagttggagatcagcctggccgacatggcgaaaccccatgtctactaaaaatacaaaaactagccaggcatggtggcacatgcctgtaatcccagctacttgggaggctgaagcaggagaatcacttgaacccaggaggcagaggctgagtgagccaagatcgtgccaatgcactccagcttgggtgagggagtgagactccgtctcaaaaaaaaaaaaaaagaaagaaaaagaaaagaaagtgatcctactggaaccatgcttactcccctccccacctcacactgtgtagaaattagtgctgtcggccaggcgcggtggctcatgcctgtaatcgcagcactttgggaggccaaggcaggcggatcacgaggtcaggagatcaagaccatcctggctaacacagtgaaaccctgtctctactaaaaatacaaaaaattagccgggcatggtggcaggcacctgtagtcccaactacttgggaggctgaggcaggagaatggcatgaacctgggaggcggagcttgcagtgagccaagatcgcgccactgcataccagcctaggtgacagagtgagactcagcaaaaaaagaaagaaagaaagaaagaaatcagtgctgtctatacttctttctgcagtgatggaaatattctgtatctgtgctgtccagtatagtagccactagctacatgtggcacttgaaacatggctggtacagttgaggaagagtggctgccatatcggacgacacagctatagattctgtcaccccaccccgagagtccagagcggggacttctgccttaggccctattcagggctgatttttacttgaacccttactgtgggaagagaaggccatgagaagttcagtctagaatgtgactccttattttctggctcccttggacactttgtgggatttagtctccctgtggaaagtattccacaagtggtgccaccaccccagctgtgagagcagctgggagctgcttttgtcatctttccctggaaagtcctgtgggctgtctcttcctcatgccttgtcccatgcttgggcatggtgtcaagcgtcaggagggagaaagggtccttatttatttatttagagagggacccttcttctgttcccaggctggagtgcagtggtgcgatctcggctcactgcaacctccgcctcctgggttcaagtgattctcctgcctcagcctcctgagtagctgagattacaggcacatgccaacatgcctggctaattttttttttttttttttttttttttttttgagatggagttgtactctcattgcccaggctggaatgtaatggcacaatctcggctcactgcaacctccacctcctggattcaagcaattctcctgtctcagcttcccaagtagctgggattacaggtgcccgccaccatgctcaactaatttttgtattttttttttagtagagacgaggtttcaccatgttggtcagactggtctcaaactcctgacctcaggtgatccacctgcctcggcctcccaaagtgctaggattacaggcatgagccaccacgcccggcctgaaagggttcttatttagtgtgcattttgacattcaatttaattccaaggtcttgtggggtcatggtttacaggatgttgatatagaaaagacttcacttaatgggccgggcgcagtggctcatgcctgtaatcccagcactttgggaggccgaggcaggcagatcaggaggtcaggagattgagaccatcctggctaacacagtgaaaccccatctctactgaaaatacaaaaaattagctgggcgtggtggcaggcacctgtagtcccagccactcggttggctgaggcaggagaatggcatgaacccgggaggcggagcttgcagtgagcagagaccatgccactgcactccagcctgggcgacagagcaagactctgtctcaagaaaaaaaaaaaaaaaacagactttacttactggaagccaaccaatgtatatttagagtaatttttcctgggctgagctgtcatttacttttgcagtatctcaagaagaagagtttacagtgtaaatatttgatgcacactttgattatatagatgaagcaaactattttcaagagctttgcaaggacttacttgtatccaaacaccattctaaaggagtcttacctacttctaaaggctggtctctacttggaaccacttgcttggccctggttcaagtcctgctgcaaacctggaagtcctgtcattgtcttcttccctccagagcagtggcacccaatctaatttttgctgtgccccagcagcccctggcactttgccctgtagactgcagacctcatgtaatgtatgttaagtccacagaaccacagaagatgatggcaagatgctcttgtgtgtgttgtgttctaggaggtggccaggtggaagtaaaatctgagaagcttgacttcaaggacagagtccagtcgaagattgggtccctggacaatatcacccacgtccctggcggaggaaataaaaag

SEQ ID NO: 19 - DNA, 20 bp, synthetic construct
  source 1..20: mol_type = other DNA
gattgggtccctggacaata

SEQ ID NO: 20 - DNA, 20 bp, synthetic construct
  source 1..20: mol_type = other DNA
gacaccactggcgacttgta

SEQ ID NO: 21 - DNA, 20 bp, synthetic construct
  source 1..20: mol_type = other DNA
actccaacagcggaaggtgg

SEQ ID NO: 22 - DNA, 20 bp, synthetic construct
  source 1..20: mol_type = other DNA
caaagcaccgaaggagtgag

SEQ ID NO: 23 - DNA, 20 bp, synthetic construct
  source 1..20: mol_type = other DNA
acgtggatgaagttggtggt

SEQ ID NO: 24 - DNA, 20 bp, synthetic construct
  source 1..20: mol_type = other DNA
cagcatcaggagtggacaga

SEQ ID NO: 25 - DNA, 20 bp, synthetic construct
  source 1..20: mol_type = other DNA
caaggtgaacgtggatgaag

SEQ ID NO: 26 - DNA, 20 bp, synthetic construct
  source 1..20: mol_type = other DNA
gtctccacatgcccagtttc

SEQ ID NO: 27 - DNA, 20 bp, synthetic construct
  source 1..20: mol_type = other DNA
gacagggtcatcccatgaag

SEQ ID NO: 28 - DNA, 20 bp, synthetic construct
  source 1..20: mol_type = other DNA
ttgtcatttcttcggcagtg

SEQ ID NO: 29 - protein, 136 aa, synthetic construct
MGRLTSSFLLLIVPAYVLSQVTLKESGPGILQPSQTLSLTCSFSGFSLSTSGMGVGWIRQPSGKGLEWLAHIWWDDVKRYNPALKSRLTISKDTSSSQVFLKIASVDTADTATYYCVRTILWMDYWGQGTSVTVSS

SEQ ID NO: 30 - DNA, 408 bp, synthetic construct
  source 1..408: mol_type = other DNA
atgggcaggcttacttcttcattcctgctactgattgtccctgcatatgtcctgtcccaggttactctgaaagagtctggccctgggatattgcagccctcccagaccctcagtctgacttgttctttctctgggttttcactgagcacttctggtatgggtgtaggctggattcgtcagccatcagggaagggtctggagtggctggcacacatttggtgggatgatgtcaagcgctataacccagccctgaagagccgactgactatctccaaggatacctccagcagccaggtattcctcaagatcgccagtgtggacactgcagatactgccacatattactgtgttcgaaccattctatggatggactactggggtcaaggaacctcagtcaccgtctcctca

SEQ ID NO: 31 - protein, 7 aa, synthetic construct
TSGMGVG

SEQ ID NO: 32 - protein, 16 aa, synthetic construct
HIWWDDVKRYNPALKS

SEQ ID NO: 33 - protein, 7 aa, synthetic construct
TILWMDY

SEQ ID NO: 34 - protein, 132 aa, synthetic construct
MDSQAQVLILLLLWVSGTCGDIVMSQSPSSLAVSAGEKVTMSCKSSQSLLNSRTRKNYLAWYQQKPGQSPKLLIYWASTRESGVPDRFTGSGSGTDFTLTISSVQAEDLAVYYCKQSYNLFTFGSGTKLEIK

SEQ ID NO: 35 - DNA, 396 bp, synthetic construct
  source 1..396: mol_type = other DNA
atggattcacaggcccaggttcttatattgctgctgctatgggtatctggtacctgtggggacattgtgatgtcacagtctccatcctccctggctgtgtcagcaggagagaaggtcactatgagctgcaaatccagtcagagtctgctcaacagtagaacccgaaagaactacttggcttggtaccagcagaaaccagggcagtctcctaaactgttgatctactgggcatccactagggaatctggggtccctgatcgcttcacaggcagtggatctgggacagatttcactctcaccatcagcagtgtgcaggctgaagacctggcagtttattactgcaagcaatcttataatctattcacgttcggctcggggacaaagttggaaataaaa

SEQ ID NO: 36 - protein, 17 aa, synthetic construct
KSSQSLLNSRTRKNYLA

SEQ ID NO: 37 - protein, 7 aa, synthetic construct
WASTRES

SEQ ID NO: 38 - protein, 8 aa, synthetic construct
KQSYNLFT